(19) United States
(12) Patent Application Publication
(10) Pub. No.: US 2013/0202687 A1
(43) Pub. Date: Aug. 8, 2013

(54) Lipid vectors delivering gene silencers

(71) Applicants: Patrizia Perri, Genova (IT); Gabriella Pagnan, Genova (IT); Fabio Pastorino, Bogliasco (GE) (IT); Mirco Ponzoni, Genova (IT)

(72) Inventors: Patrizia Perri, Genova (IT); Gabriella Pagnan, Genova (IT); Fabio Pastorino, Bogliasco (GE) (IT); Mirco Ponzoni, Genova (IT)

(73) Assignee: ISTITUTO GIANNINA GASLINI, Genova (IT)

(21) Appl. No.: 13/879,790

(22) PCT Filed: Oct. 14, 2011
(86) PCT No.: PCT/IB11/54571

§ 371 Date: Apr. 16, 2013

(30) Foreign Application Priority Data
Oct. 18, 2010 (IT) GE2010A000114

(51) Int. Cl. C07H 21/02

(52) U.S. Cl. CPC C07H 21/02
USPC 424/450; 514/44 A; 536/24.5; 435/375

(57) ABSTRACT

Lipid vector delivering gene silencers which vector carries on its external surface at least one recognizing molecule directed against a cellular target molecule. The recognizing molecule is directed against GD₂ disialoganglioside epitope, a tumor-associated membrane antigen expressed abundantly by neuroectodermal tumor cells, and which lipid vector contains as therapeutic agent double-stranded RNA synthetic oligonucleotides such as siRNAs (small interfering RNAs), complementary to mRNAs (messenger RNAs) coding for at least one oncogene such to obtain silencing, that is the selectively decreased expression of the oncogene, in target cells.

The present invention relates to a lipid vector delivering gene silencers as therapeutic agent in target cells.

Neuroblastoma (NB) is the most common extracranial solid tumor of childhood.

Patients with high risk neuroblastoma are currently treated with radiotherapy, intensive chemotherapy, autologous stem cells, 13-cis-retinoid acid, or monoclonal antibodies against proteins specific to neuroblastoma.

The rate of 3-year disease-free survival is 34% (Maris et al., 2007; Matthay et al., 2009). In addition, because of limiting factors related to intensive induction therapy such as acute and chronic toxicity and the development of secondary tumors (Gesundheit et al., 2007), the prognosis for patients with recurrent or refractory high-risk neuroblastoma remains poor.

The development of novel antitumoral strategies is therefore essential to improve the low rate of survival of patients at high risk.

Notwithstanding the application of therapeutic protocols traditionally used in the clinical practice, such as surgery, radiotherapy, high-dose chemotherapy and hematopoieticstem cell transplantation, long-term survival of patients with advanced stage neuroblastoma has not satisfactorily improved.

In most cases, the use of high concentrations of antitumoral drugs leads to diffuse systemic toxicity.

Liposomal encapsulation of antitumoral agents induces a reduction of side effects of the entrapped drug improving its therapeutic efficacy.

In fact, it is well known that liposomes, i.e. vesicles with one or more lipid layers, can be used as excellent vectors for drug administration: they are able to increase the therapeutic efficacy of the encapsulated drug and simultaneously reduce its toxic effects thanks to more specific targeting.

Thanks to their simple formulation, due to the fact that they are structurally similar to biological membranes, liposomes guarantee biodegradability, biocompatibility and absence of immunogenicity; in addition, the possibility of modifying their surface allows their pharmacokinetic parameters to be modulated.

In particular, in order to reduce macrophage uptake, liposome surface can be coated with hydrophilic molecules, typically polyethylene glycols (PEG). Such coating allows them to have a stable circulation for days and guarantees their consequent extravasation through fenestrations of blood neovessels developed within the tumor mass, assuring passive targeting to the tumoral tissue.

In addition, the external surface of the lipid covering can be decorated with antibodies specific for tumor-associated antigens: immunoliposomes thus allow the therapeutic index of the encapsulated cytotoxic drugs to increase while minimizing their systemic toxicity thanks to the fact that their selectivity can be improved by coating their external surface with antibodies against tumor-associated antigens.

Disialoganglioside GD₂ is a tumor-associated membrane antigen abundantly expressed in human neuroblastomas.

Disialoganglioside GD₂ is a tumor-associated antigen largely expressed by neuroectoderm-derived cells, such as neuroblastoma and melanoma, whereas in normal cells it is mildly expressed only by cerebellum and peripheral nerves.

Immunoliposomes can form lipopolyplexes to be used to deliver antisense oligonucleotides to tumor cells in order to modulate their oncogene expression.

To prevent messenger RNA (mRNA) translation into protein, the use of antisense oligonucleotides is well known, they are short DNA or RNA sequences that are synthesized for sequence-specific coupling to RNA of a specific target molecule. This binding prevents mRNA translation into protein.

It is also well known that RNA interference technique can be used for protein ablation. The technique consists in the introduction into the cell of short double-stranded RNA molecules called siRNAs (small interfering RNAs), whose antisense strand presents a sequence complementary to that of the mRNA of the gene to be silenced. siRNAs are associated with the enzyme complex called RISC(RNA-induced silencing complex), which is activated by this binding, the two strands unroll, the antisense strand couples in a highly sequence-specific fashion to target mRNA and the enzyme complex degrades the mRNA molecule, thus preventing the synthesis of the corresponding protein.

The molecules mediating RNA interference (RNAi) present several advantages compared to other small inhibitory molecules, antibodies, and other biological agents, both for molecular biology studies, aimed at identifying the target genes and their signal transmission pathways, and for drug development.

Among the advantages of RNAi there is the extremely high selectivity, due to sequence-specific gene silencing guaranteeing the knock-down of both wild-type and mutated gene transcripts, and their low toxicity.

Notwithstanding the considerable potential of RNAi for the treatment of cancer, several obstacles must be overcome before exogenous siRNA can be largely used in therapies against cancer.

In addition, for in vivo studies, several obstacles still remain to be overcome so that siRNA release to target cells is specific and effective and siRNA result non toxic: this represents the major barrier limiting the therapeutic applications of siRNA.

The progressive use of RNAi technology is hindered by the following:
lability of siRNA in organic fluids which thus leads to a strong and rapid degradation by serum nuclease,
poor membrane permeability limiting its cellular uptake,
rapid glomerular filtration leading to poor pharmacokinetics,
possible activation of siRNA-mediated innate immune response,
need to obtain an effective intracellular release in target cells in vivo avoiding non specific distribution.

It is also known that Anaplastic Lymphoma Kinase, ALK, is a tyrosine kinase receptor involved in the pathogenesis of different tumor types in humans, in particular it is a tyrosine kinase receptor involved in the pathogenesis of a subset of human lymphomas called Anaplastic Large Cell Lymphomas (ALCL).

ALK has recently proved to contribute to the pathogenesis of human neuroblastoma through oncogenic events as gene mutations, gene amplifications and/or deregulated gene expression.

ALK was initially described as translocated in the anaplastic large cell lymphoma, then it was identified as an oncogene in a fraction of lung tumors while, more recently, point mutations have been described in familial or sporadic neuroblastoma.

The recent identification of germline mutations or somatic mutations acquired in the gene coding for ALK opens up for the first time new perspectives in the development of innovative gene therapies (e.g. having at least one oncogene as target) for neuroblastoma, a severe pediatric tumor accounting for 15% of cancer-related childhood mortality (Cohn et al., 2009; Maris et al., 2007).

It is well known that ALK has a physiological role in neuronal development which is involved in cancer pathogenesis, especially of lymphomas but also of ectodermal, myofibroblastic or neuroblastic solid tumors (Chiarle et al., 2008; Palmer et al., 2009; Webb et al., 2009).

About 11-12% of neuroblastomas leads to a non-synonymous sequence variation in conserved positions in the kinase domain.

It is worth noting that the most frequent ALK mutant proteins, which carry F1174L or R1275Q amino acid substitutions, demonstrate a gain of function in kinase activity (Chen et al., 2008; Gorge et al., 2008; Janoueix-Lerosey et al., 2008; Mosse et al., 2008).

Many cases of neuroblastoma show high levels of ALK expression. It was also demonstrated that this ALK over-expression plays a pathogenetic role in neuroblastoma.

In addition, ALK resulted to be highly expressed in rhabdomyosarcomas.

In any case, only 3-4% of neuroblastoma cases lead to extended ALK amplification while 17-23% present lower levels of increase in ALK gene copies (Caren et al., 2008; Chen et al., 2008; George et al., 2008; Janoueix-Lerosey et al., 2008; Mosse et al., 2008; Passoni et al., 2009).

Over-expression of both mutated and wild-type (WT) ALK proteins induces a constitutive kinase activity.

On the other hand, the knock-down of ALK expression in cell lines leads to a marked decrease in cell proliferation which clearly attributes to ALK a critical role in the development of neuroblastoma.

Mutations and amplifications of ALK as well as gene over-expression were found significantly correlated with other unfavourable characteristics of poor prognosis in advanced or metastatic disease compared to localized tumors (Caren et al., 2008; Chen et al., 2008; George et al., 2008; Janoueix-Lerosey et al., 2008; Mosse et al., 2008; Palmer et al., 2009; Passoni et al., 2009).

ALK gene can therefore be an interesting target for the development of innovative antitumoral therapies, in particular against ALCLs, lung cancer, and neuroblastoma.

The most obvious approach to block ALK activity is the development of small inhibitory molecules: the development of small molecules able to inhibit ALK kinase activity represents an attractive therapeutic instrument for the treatment of ALK-dependent neuroblastomas as well as other ALK-dependent tumors in general (Chiarle et al., 2008).

At present, the efficacy of ALK inhibitors in neuroblastoma has been demonstrated only in vitro in some but not all cell lines carrying ALK mutations, in particular in cell lines carrying activating point mutation F1174L, while cells carrying R1275Q mutation are resistant to ALK inhibitors (George et al., 2008; Webb et al., 2009). There are no data on the efficacy of these inhibitors in vivo or in neuroblastoma cell lines carrying ALK WT (wild type) or amplified gene.

In addition, known kinase inhibitors lead to forms of drug resistance in the treated population.

Objective of this invention is to make the action of the used antitumoral agent more selective and potent through its intracellular release in target cells by means of a specific membrane binding and subsequent cellular uptake, i.e. the entry of the agent into the tumor cell, thus by avoiding non-specific distribution of the agent. Another objective is to obtain the release of a anti-tumor agent with optimal pharmacokinetic characteristics, without acute and chronic toxic effects, in order to have an increased therapeutic efficacy in vivo.

The above-mentioned objective is obtained through a lipid vector for the transfer into the target cells of at least one therapeutic agent such as a gene silencer, which lipid vector carries on its external surface at least a recognizing molecule directed against a cellular target molecule, in particular the recognizing molecule is directed against the epitope of disialoganglioside GD₂, a tumor-associated membrane antigen expressed abundantly by neuroectodermal tumor cells, and which lipid vector contains as therapeutic agent double-stranded RNA synthetic oligonucleotides such as siRNA (small interfering RNA), complementary to mRNA (messenger RNA) coding for at least one gene with oncogenic properties in order to obtain gene silencing, i.e. the selective decrease of expression of said gene in target cells.

In particular, the lipid vector allows the nucleic acids to be transfected, in particular oligonucleotides, to target cells.

Preferably, the lipid vector is a liposome that, to obtain active targeting (direction against a specific target) carries on its surface at least a recognizing molecule that can be defined as targeting partner, such as antibodies or antibody fragments, vitamins, peptides, which molecule recognizes tumor-associated molecules, i.e. specific membrane molecules of tumor cells and which liposome contains oligonucleotide sequences, in particular siRNA (small interfering RNA), specific for one gene.

shRNA (short hairpin RNA) and siRNA can be designed and synthesized starting from the known sequence of mRNA (messenger RNA) of the target gene, e.g. ALK gene.

Protocols and design services of siRNA or shRNA are available on line, for instance in the following websites: http://genelink.com/sirna/shRNAi.asp or http://www.ambion.com/techlib/misc/psilencer-converter.html. People skilled in that art have no difficulty in designing appropriate siRNAs to perform the invention.

The presence of the recognizing molecule allows the lipid vector to be bound in a high specific manner to the cell that expresses on its surface the specific target protein, increasing the amount of oligonucleotides released in the target cell and making release highly specific and efficient.

Preferably, the recognizing molecule has as preferentially target, molecules expressed on tumor cell surface, being siRNA contained in the liposome specific for gene silencing of oncogenes.

The term recognizing molecule must be taken in its broadest sense and therefore includes a ligand, a natural or synthetic antibody, or a fragment thereof, and in general a molecule able to recognize and bind to a membrane molecule of the target cell, such as a receptor, a marker, an antigen or a membrane component of the target cell.

According to the present invention, the recognizing molecule definable as targeting partner, is an antibody or an antibody fragment directed against the disialoganglioside GD₂, a tumor-associated fragment highly expressed by neuroectodermal tumor cells.

The definition non viral lipid vector means a closed vesicle composed of one or more lipid layers.

The liposome of the invention entraps nucleic acids specific for oncogene silencing at post-transcriptional level, i.e. through decrease of gene expression (down-modulation), in particular ALK-specific siRNA, namely it contains a therapeutic agent inside the liposome.

Preferably, the liposome is a cationic liposome coated with neutral lipids (coated cationic liposome, CCL) allowing nucleic acids with negative charges to be condensed in association with lipids with positive charges, forming the so-called core. This core is subsequently coated with a neutral lipid layer to avoid systemic toxicity due to cationic lipids and can be delivered specifically to tumor cells thanks to the molecule that recognizes a specific target of tumor cells which is necessary to allow nucleic acids to go through cell membranes via selective receptor.

Patent GB 0312309.8 describes examples of liposomal formulations.

Examples of cationic lipids are the following: DOTAP, DMRIE, DORIE, DOTMA, DDAB, and DC-chol.

In particular, as described later on, siRNA were complexed with cationic lipids and coated with neutral lipids.

Coated cationic liposomes (CCLs) described hereafter are stable and efficient transporters able to contain more than 90% of encapsulated siRNA molecules and to protect siRNAs against their rapid degradation by serum nucleases.

Preferably, as described in detail below, anti-GD₂ monoclonal antibody is coupled to maleimide (MAL) terminal of DSPE-PEG₂₀₀₀ lipid (DSPE-PEG₂₀₀₀-MAL), a lipid which is part of the neutral external secondary layer, to allow the new particle entrapping siRNA to target GD₂-positive cells.

As antibodies directed against GD₂, it is possible to use the so-called chimeric antibodies which are more similar to human antibodies or to humanized antibodies.

In a preferred embodiment, CCLs have as recognizing molecule an anti-GD₂ antibody and siRNA are specific for ALK oncogene.

Among the specific antigens found on neuroblastoma cell membrane, disialoganglioside GD₂ is an attractive target for immunoliposomal therapy of neuroectodermal tumors, since it is expressed extensively by these tumor cells compared to non malignant tissues.

Liposomal encapsulation of siRNAs, as anti-tumor agents, induces a reduction of side effects while improving their therapeutic efficacy, since it allows a highly specific and efficient release.

ALK is a tyrosine kinase receptor contributing to the pathogenesis of human neuroblastoma through oncogenic events such as mutations, amplifications and/or deregulated expression.

ALK plays a central role in neuroblastoma progression and tumor angiogenesis, and therefore is an important target for an innovative therapeutic strategy.

Design of siRNAs specific for ALK oncogene guarantees high sequence specificity, irrespective of gene status (wild-type, mutated or amplified), and an effective gene knock-down without off-target effects.

Treatment with siRNAs for ALK entrapped in nanoparticles having GD₂ as target leads to tumor growth inhibition and angiogenesis in neuroblastoma.

Solid tumors involve the development of new blood vessels (angiogenesis) supporting tumor growth and, consequently, the possibility of targeting genes involved in tumor angiogenesis allows anti-angiogenic therapies for the treatment of cancer to be developed.

Considering the frequent development of drug resistance to oncogenic kinase inhibitors, especially after prolonged treatments, objective of the present invention is to develop the association of one or more kinase inhibitors with non-viral vectors, in particular liposomes (CCLs) that entrap siRNA directed against ALK, a synergy that can prevent drug resistance in patients with neuroblastoma.

Subject of the present invention is therefore a formulation comprising the above-described lipid vector in combination with a further natural or synthetic therapeutic agent, either free or contained within viral and/or non-viral vectors, such as an antibody, a protein inhibitor, a chemotherapeutic agent.

For instance, kinase inhibitors or EGFR inhibitors.

According to one embodiment, it is possible to provide a combination of at least two different types of lipid vectors in which at least one of the vectors comprises a different recognizing molecule (targeting partner) directed against a molecule different from GD₂ and/or containing as therapeutic agent an agent different from siRNAs.

For instance, as therapeutic agent different from siRNA, in particular different from ALK-specific siRNA, it is possible to use: single-stranded antisense oligonucleotides provided with CpG islands, microRNAs (miRNAs) or miRNA mimics or miRNA antagonists, an anticancer drug such as the chemotherapeutic agent doxorubicin, an anti-inflammatory drug or an anti-angiogenic drug, a protein inhibitor, for instance an inhibitor of ALK kinase activity.

It is clearly possible that the liposomal formulation which is the subject of the present invention is used to transfer at least any one of the agents within the cell, preferably sequence-specific synthetic oligonucleotides, directed against any target gene.

Further object of the present invention is to provide a pharmaceutical composition comprising the non-viral lipid vector of the present invention associated with pharmacologically acceptable solid and/or fluid excipients.

The lipid vector of the present invention can therefore be used for the preparation of a drug for the treatment of cancer and/or for the treatment of associated phenomena such as neoangiogenesis and/or metastatization.

The therapeutic agent contained in the lipid vector of the present invention can be single, combined with one or more therapeutic agents, free or contained in viral or non-viral vectors, and/or in the form of a pharmaceutical composition and pharmaceutical kit for the treatment of tumors, in particular for the treatment of neuroectodermal tumors such as neuroblastoma and melanoma, and/or associated phenomena.

A pharmaceutical formulation is a composition that comprises or is composed of a therapeutically effective quantity, i.e. a quantity able to obtain the desired effects, of an active principle. Preferably, the composition comprises an excipient, a diluent, a transporter, or a pharmacologically acceptable combination thereof, i.e. excipients making it possible to obtain a formulation allowing patients to be administered in the safest and most appropriate way, for instance administration through tablets, pills, syrup, granules, injection fluids or the like.

In particular, the pharmaceutical composition of the present invention can be prepared in a formulation for intravenous or intratissutal injection, so that it is possible to administer the lipid vector containing siRNA for knocking down preferably ALK gene alone or in combination with other therapeutic agents and/or pharmacologically acceptable excipients. The present invention relates also to a kit for the administration of a vector or a composition for the treatment of cancer, in particular of neuroectodermal tumors, and/or the treatment of associated phenomena such as angiogenesis and/or metastatization, which kit comprises a vector or a composition according to the described and claimed characteristics. Subject of this invention is also the use of a lipid vector or the use of a composition comprising at least said vector for first use in medical treatment of tumors, in particular neuroectodermal tumors such as neuroblastoma, melanoma, neurosarcoma, and/or associated phenomena such as angiogenesis and/or metastatization, in which said vector or said composition are implemented as described in the present application.

Subject of the present invention is also the use of a lipid vector or the use of a composition comprising at least said vector for the implementation of a medicament for the treatment of tumors, in particular neuroectodermal tumors such as neuroblastoma, melanoma, neurosarcoma and/or associated phenomena as angiogenesis and/or metastatization, in which said vector or said composition are implemented as described in the present application.

In addition, subject of the present invention is the use of siRNA for gene silencing of ALK gene in any mutation status in the patient's tumor to hinder its oncogenic activity. In fact, molecules mediating RNA interference or RNAi proved effective on both mutated and wild-type ALK transcripts besides having an extremely high sequence specificity.

One aspect of the present invention is to also to provide a method for the treatment of cancer and/or for the treatment of one or more tumor-associated conditions such as neoangiogenesis and/or metastatization, providing the administration of the lipid vector and/or of the composition, which are the subjects of the present invention, alone or in combination with other therapeutic agents or compounds.

This method can be used for the treatment of lymphomas, lung cancer, rhabdomyosarcomas, but also ectodermal, myofibroblastic or neuroblastic solid tumors.

Cancer can be of neuroectodermal origin, such as neuroblastoma or melanoma.

The method for the treatment of cancer thus comprises the administration of a certain dose of therapeutic agent contained in the lipid vector or in the composition that constitute the subject of this invention.

The administration of said lipid vector and/or of said composition, alone or in combination with other therapeutic agents or compounds, can be intratumoral or intravenous or subcutaneous.

Therefore a new strategy to obtain an antitumoral response in neuroblastoma is described.

As detailed further on, the use of sequence-specific siRNA for ALK oncogene in any gene status, wild-type, mutated, amplified, inhibits cell proliferation and/or induces apoptosis and/or decreases the density of blood vessels in antitumoral therapies, in particular in neuroectodermal tumors such as neuroblastoma and melanoma.

siRNAs can thus be used for the production of a medicament able to inhibit cell growth and/or to induce apoptosis of tumor cells in a patient, human or animal, and/or decrease the density of blood vessels.

The medicament can be administered intratumorally, intravenously or subcutaneously.

siRNAs that are sequence specific for ALK lead to:
inhibition of MAP-kinase or MAPK pathway and consequent decrease of ERK1/2 kinase phosphorylation,
inhibition of HIF-1α signal pathway and consequent decrease in the production of Vascular Endothelium Growth Factor (VEGF) and its receptor VEGFR-2,
inhibition of endothelial and perivascular cells highlighted respectively with CD34 and SMA proteins and MMP-2 and MMP-9 metalloproteinases.

In particular, the lipid formulation described here makes it possible to obtain:
protection of siRNAs, microRNAs (miRNAs), miRNA mimics, miRNA antagonists against degradation by nucleases and increase in the stability and therefore in the activity of siRNAs, microRNAs (miRNAs), miRNA mimics, miRNA antagonists
increased recognition and specific cellular uptake,
avoidance of the activation of microRNAs (miRNAs), miRNA mimics, miRNA antagonists, siRNA-mediated innate immune response, in order to obtain a better pharmacokinetics and a highly sequence-specific gene silencing, with particular reference to an oncogene in specific target cells.

In fact, it has been demonstrated that, irrespective of ALK mutational status, ALK kinase activity heavily contributes to malignant growth of neuroblastoma.

ALK is an oncogene that controls cellular growth of neuroblastoma in vitro and can be a valid therapeutic target in vivo.

As detailed further on, ALK was knocked down in vitro and in vivo through stable transfections of different neuroblastoma cells with an inducible lentiviral vector expressing short hairpin RNA (shRNA) against ALK or using liposomes directed against neuroblastoma to release systematically small interfering RNA (siRNA) directed against ALK, used as a safer alternative to shRNA-mediated adenoviral or lentiviral therapy to decrease neuroblastoma growth.

ALK knock-down through inducible short hairpin RNA (shRNA) in neuroblastoma cells with a wild-type or mutated ALK genotype induced cell cycle arrest and death of tumor cells in vitro.

In order to target selectively neuroblastoma cells in vivo, the subject of the present invention is a liposomal formulation containing small interfering RNA (siRNA) directed against ALK, which liposomes bind selectively disialoganglioside GD₂ which is present on neuroblastoma cell surface.

It is therefore possible to have a method for the induction of cell proliferation arrest, apoptosis and inhibition of angiogenesis which involves the transfer into specific target cells of molecules mediating RNA interference or RNAi, in particular siRNA specific for ALK gene through a non-viral lipid vector and/or a composition comprising this lipid vector as described.

GD₂-targeted ALK-siRNA liposomes, i.e. liposomes containing siRNA specific for ALK gene and directed against GD₂ (with antibodies specifically directed against GD₂ membrane antigen of target cells), lead to an increased stability of siRNA (thus resulting active for a longer time) and an increase in their plasmatic concentration. As a consequence, these characteristics improve liposome binding to the target cell, its uptake inside the cell, gene silencing and induction of apoptosis specifically in neuroblastoma cells.

In neuroblastoma xenografts, intratumoral injections of GD₂-targeted liposomes identified by the abbreviation TL (GD₂-targeted liposomes, i.e. liposomes with recognizing molecules for disialoganglioside of target cells) inhibit cell proliferation, induce apoptosis and the decreased density of blood vessels, as the expression in the extracellular matrix of MMP-2 and MMP-9 metalloproteinases.

Finally, intravenous administration (i.v.) of liposomes directed against GD₂ (GD₂-targeted) showed antitumoral activity in neuroblastoma due to specific knock-down of ALK, in the absence of toxic side effects upon repeated administrations.

Thanks to this invention, ALK ablation through RNA interference or RNAi in vitro and in vivo, irrespective of its gene status (WT i.e. wild-type, mutated, amplified or overexpressed), block tumor growth, induces apoptosis and inhibits angiogenesis in neuroblastoma, as demonstrated further on by CD34 (transmembrane protein), SMA and VEGF/VEGFR-2 (Vascular Endothelial Growth Factor/Receptor 2) markers.

Therefore, in this invention, an antibody having as target GD₂ expressed on neuroblastoma cell membranes was coupled with a liposomal formulation, in particular a cationic liposome coated with neutral lipids and containing siRNA, and was used to treat subcutaneous and pseudometastatic neuroblastoma xenografts in mice. Even though studies were performed in mouse models, the scope of this invention may be applied in human and animal subjects.

### DESCRIPTION OF FIGURES

FIG. 1. ALK-shRNA inhibits cellular growth and induces cell death of human neuroblastoma cell lines.

(A) SK-N-BE2 cells (neuroblastoma cell line with wild-type ALK) and SH-SY5Y cells (neuroblastoma cell line with mutated ALK) were transduced with two different inducible lentiviral constructs for specific shRNA against ALK (A5) and a control sequence (M5). Native cells and cells treated with 1 μg/ml of doxycycline for 72 hours were then lyzed and tested by immunoblotting with the indicated antibodies.

(B) Inducible SK-N-BE2 and SH-SY5Y described in (A) were mixed at a 1:1 ratio with wild-type (WT) cells and then cultured in the presence of 1 μg/ml of doxycyline to induce eGFP (Enhanced Green Fluorescent Protein) expression and to decrease ALK expression (down-modulation). Growth rate was evaluated by flow cytometry, FACS (Fluorescence Activated Cell Sorting), which records over time the percentages of eGFP-positive cells.

(C) Inducible SK-N-BE2 and SH-SY5Y cells were treated as described at point (A), then the percentages of apoptotic cells were measured by TMRM (tetramethylrhodamine methyl ester) staining.

(D) SK-N-BE2 and SH-SY5Y cells, inducible for A5 or M5, were treated with doxycycline and analyzed by Western Blot with specific antibodies.

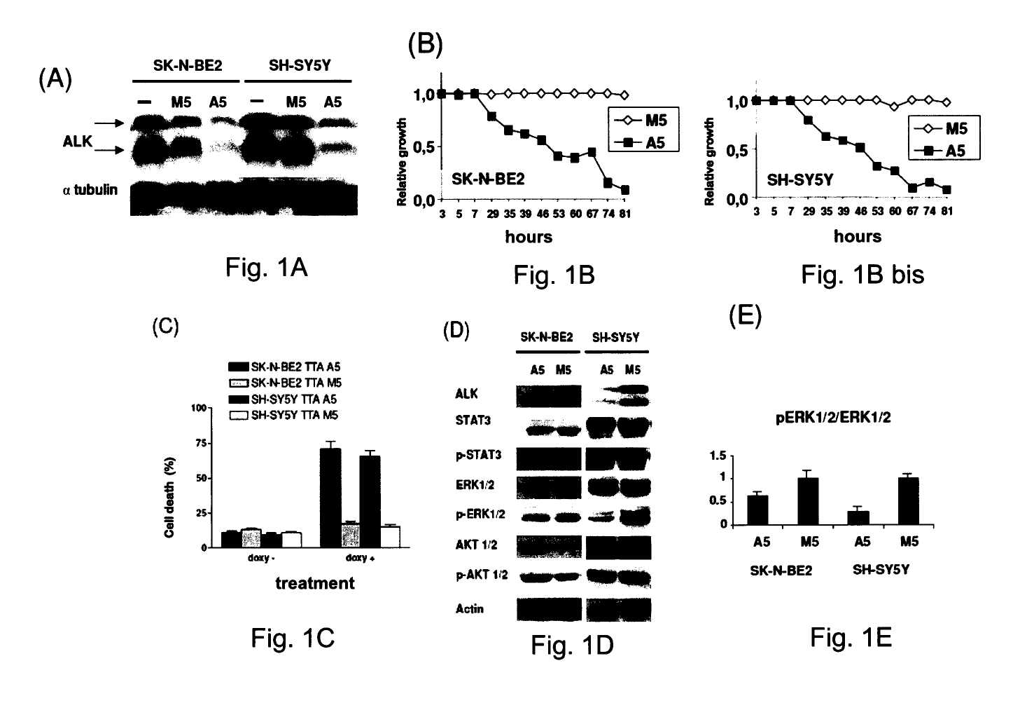

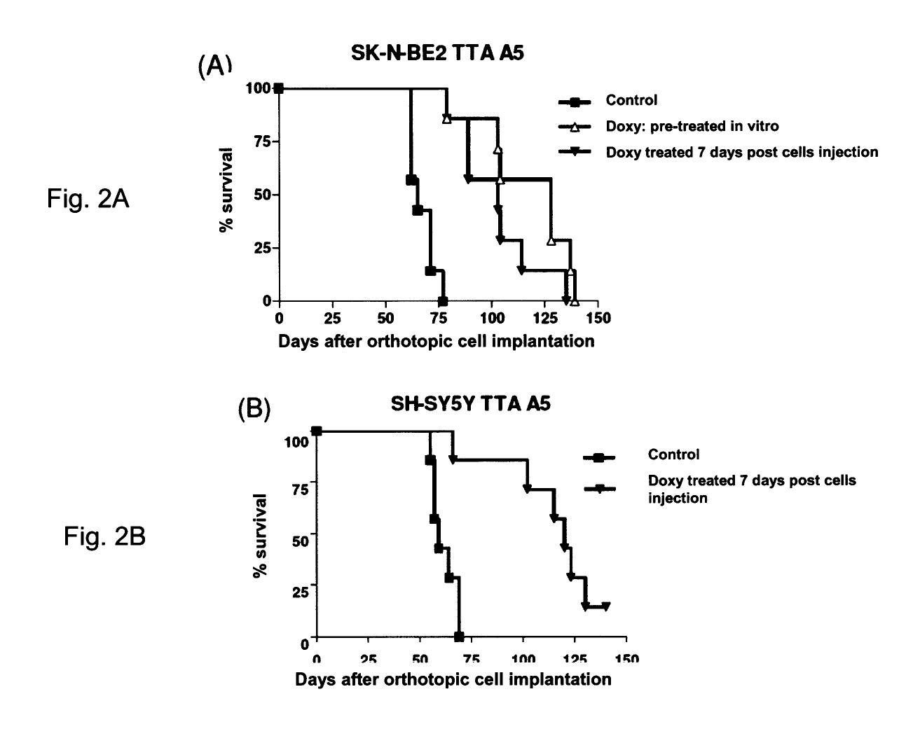

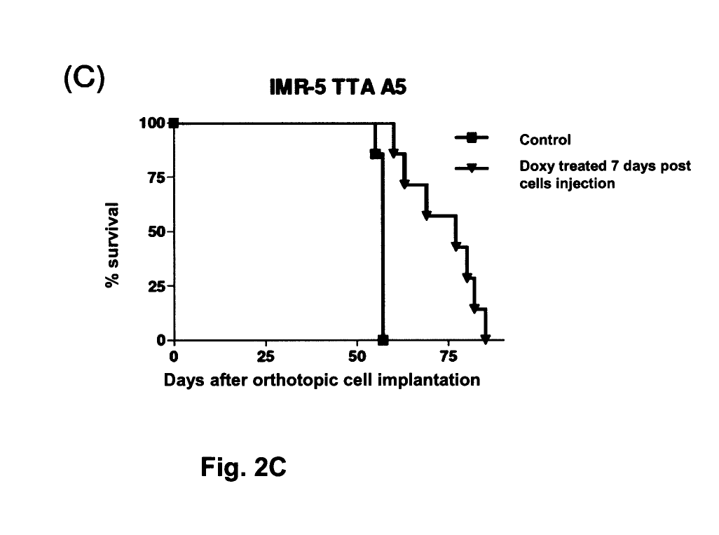

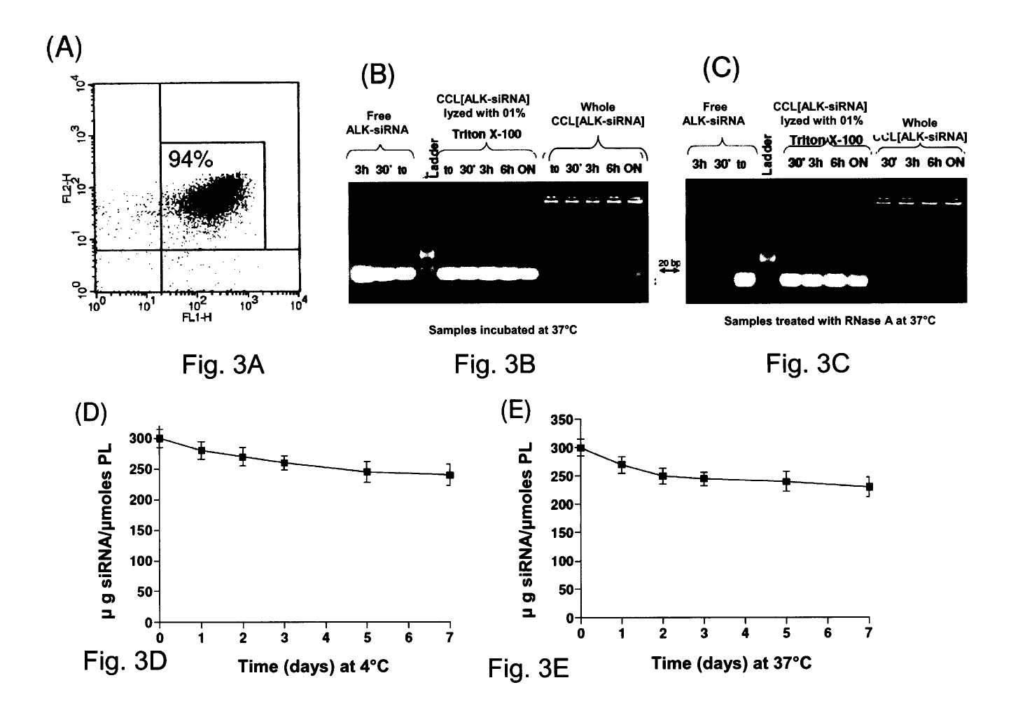

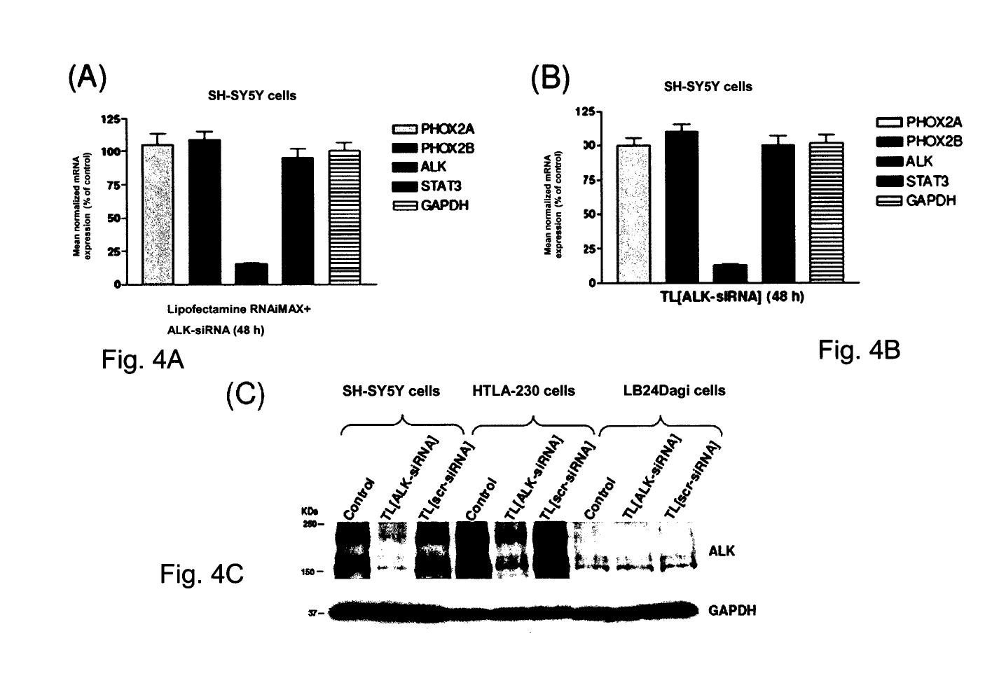

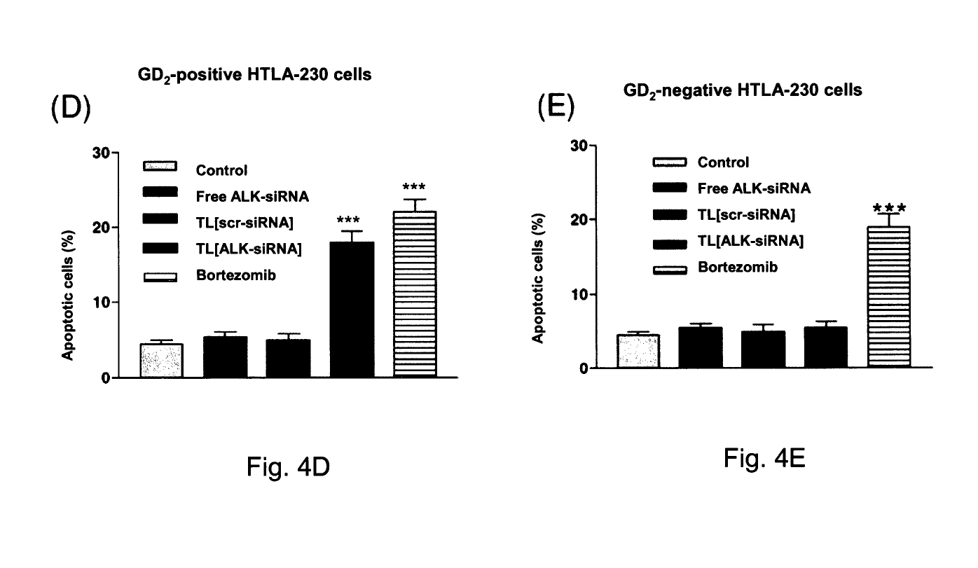

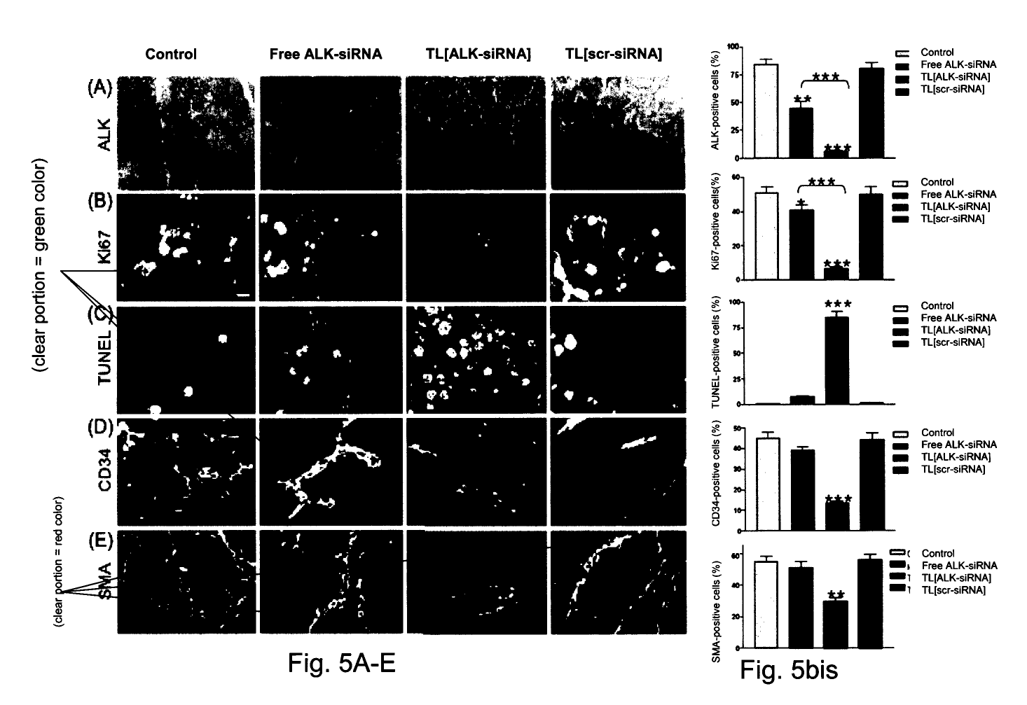

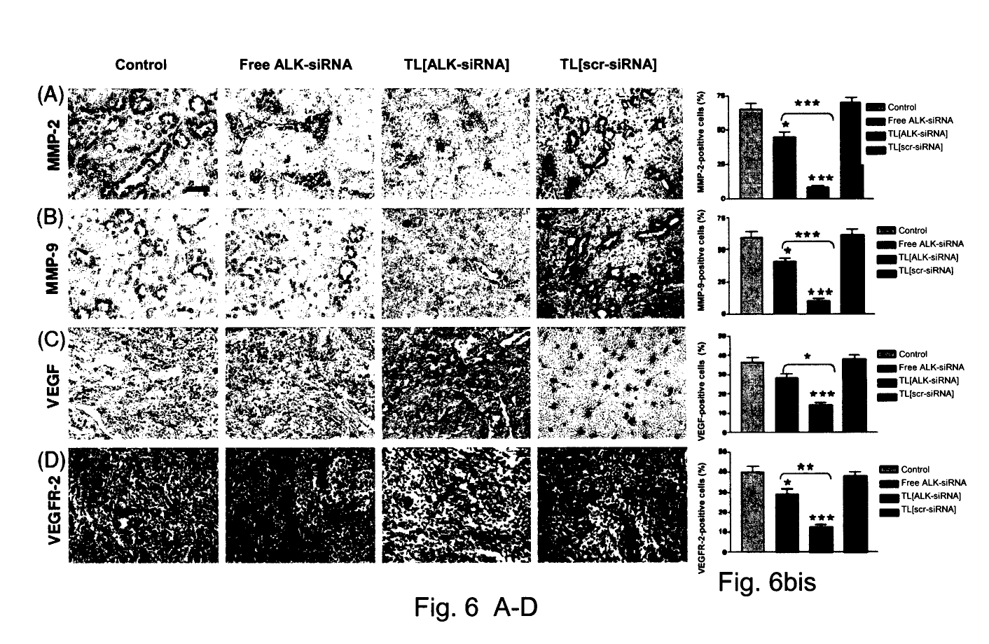

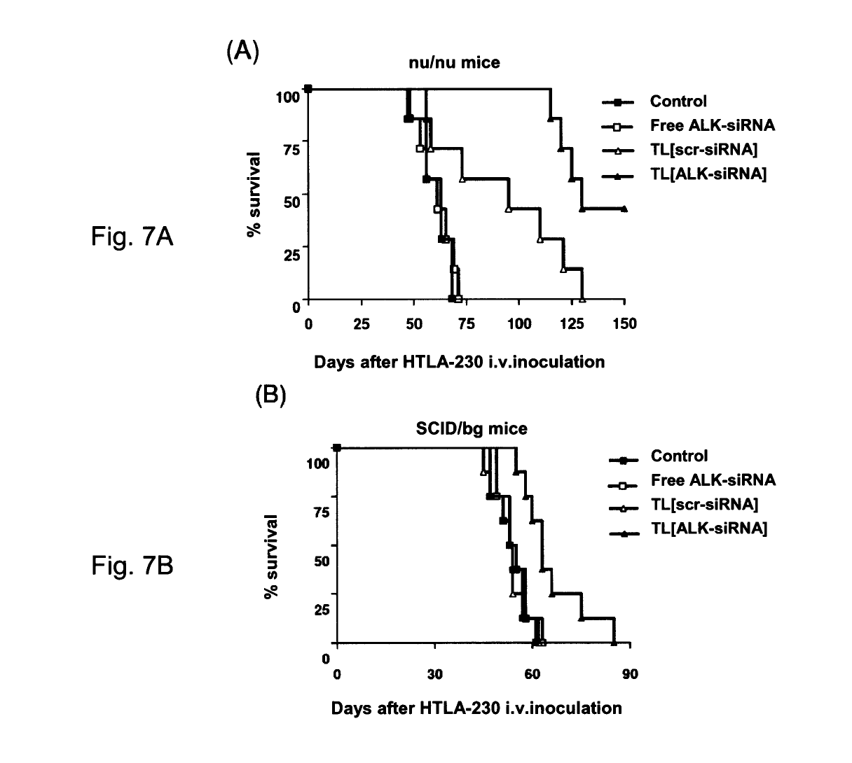

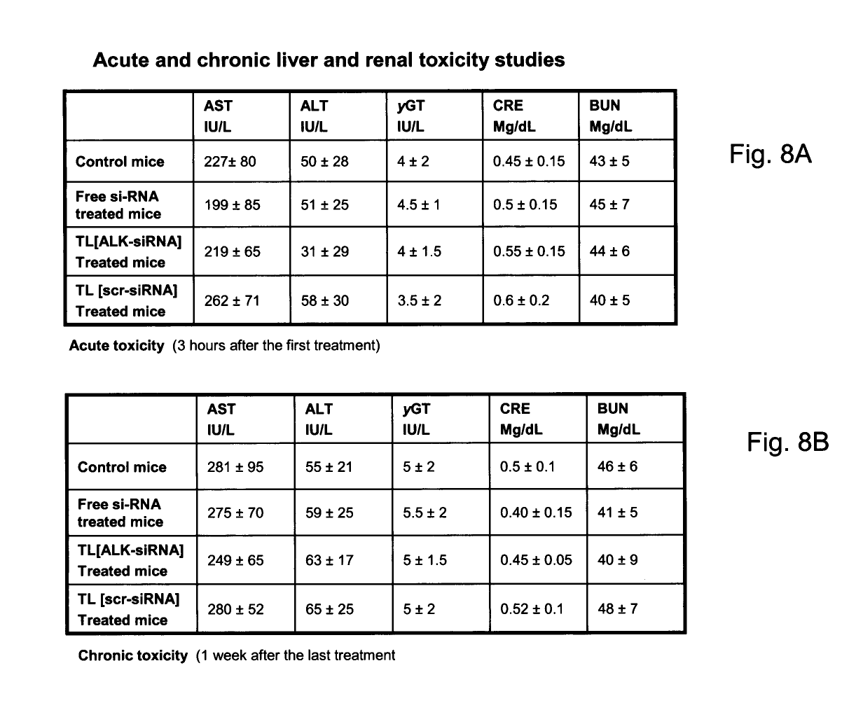

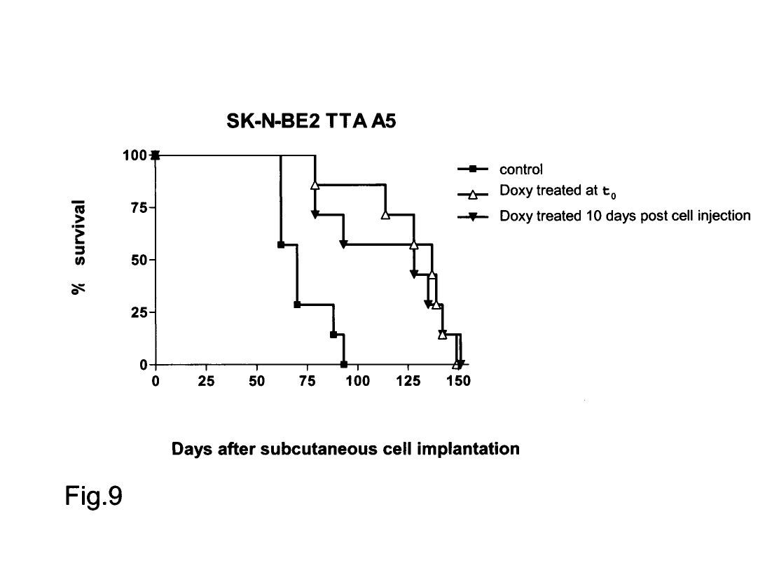

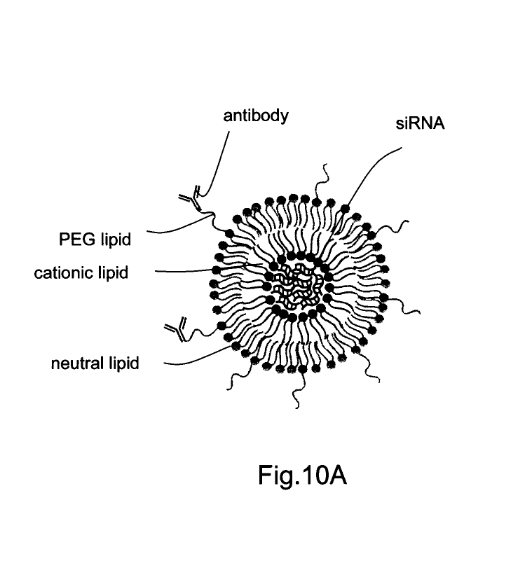

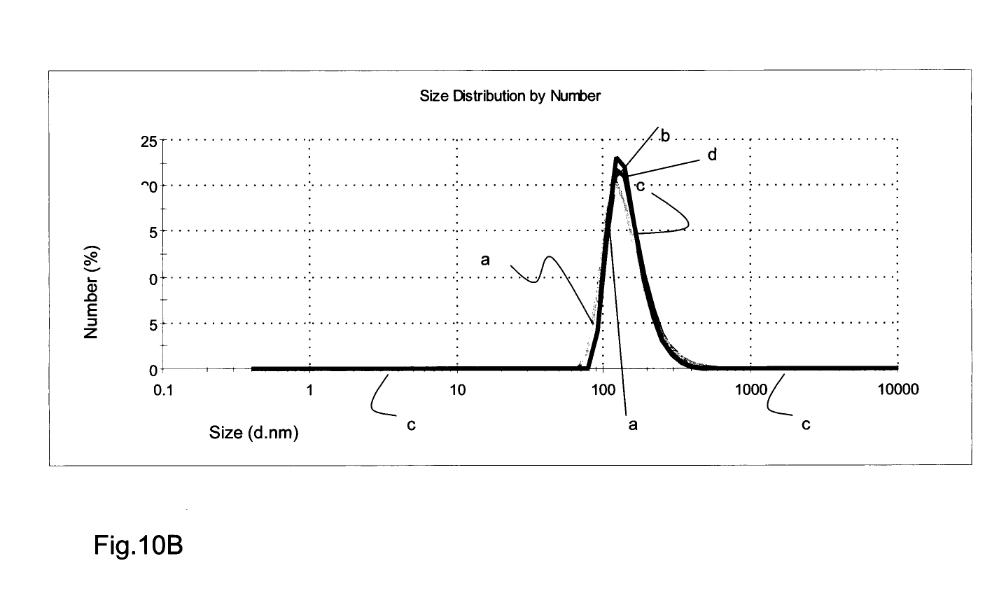

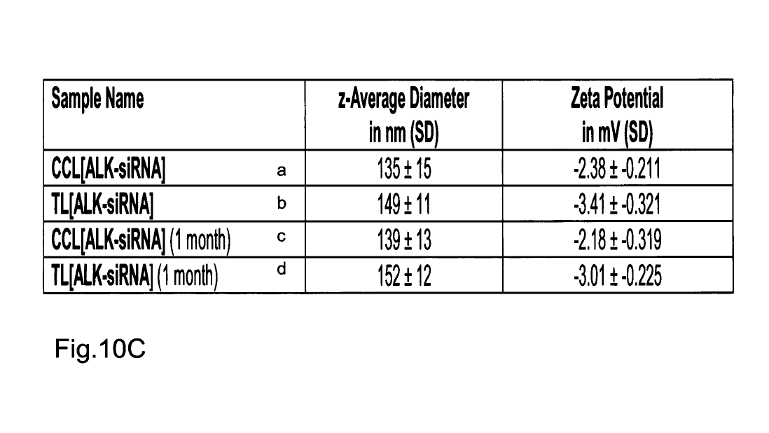

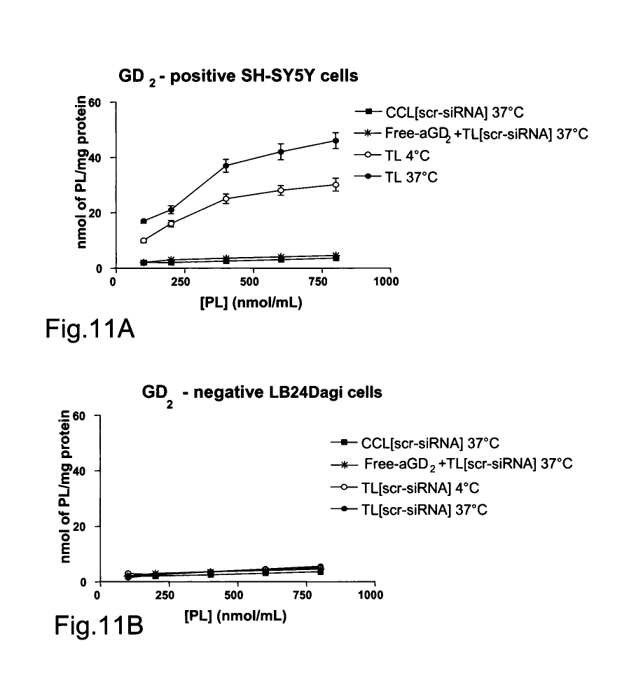

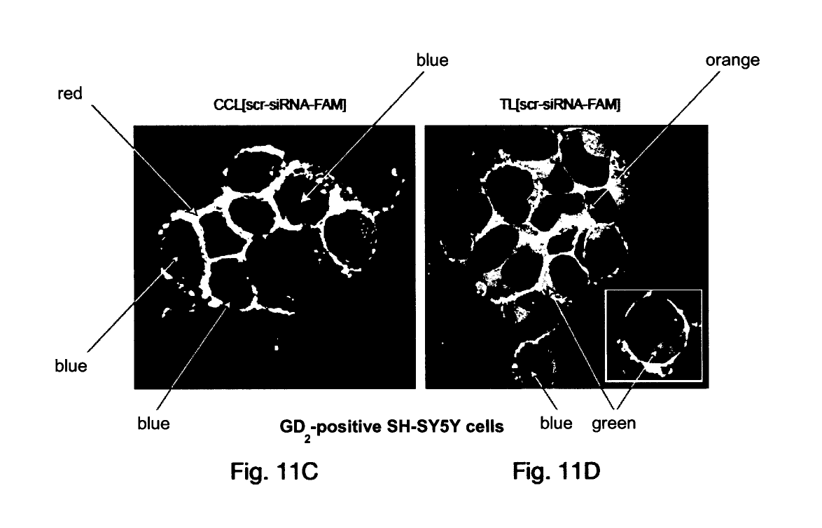

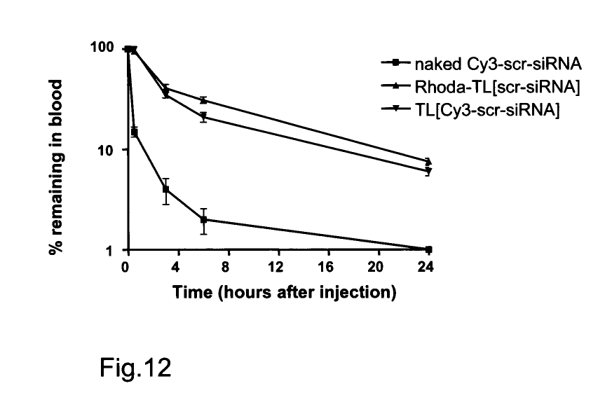

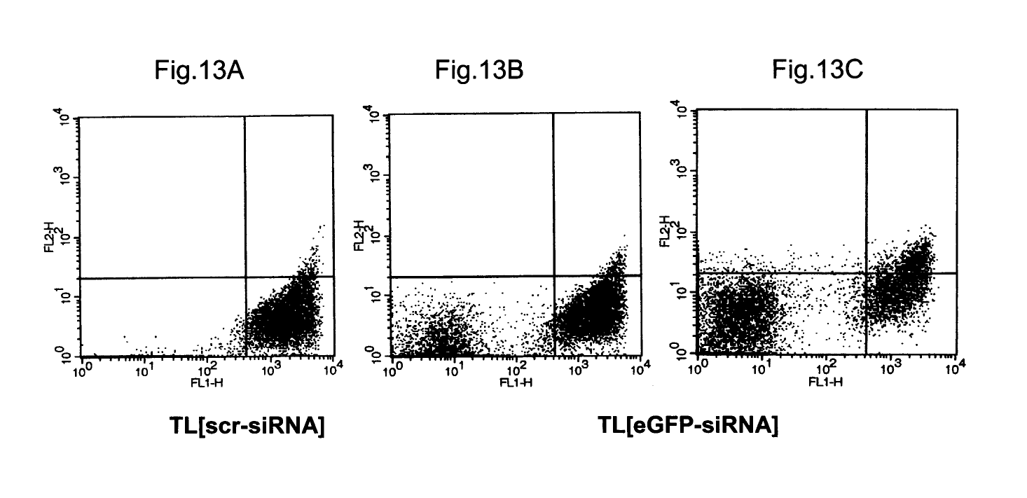

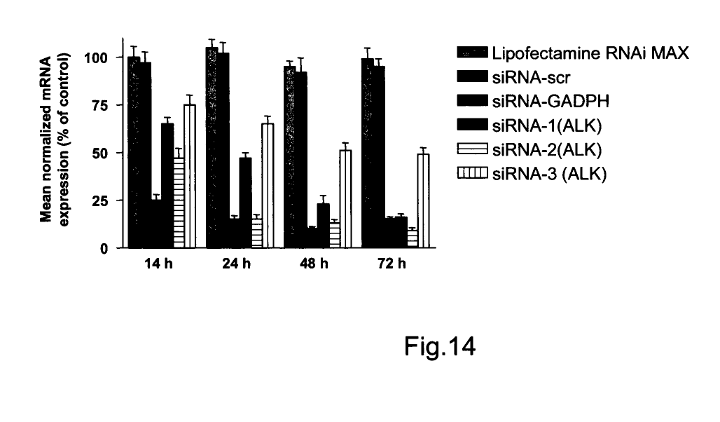

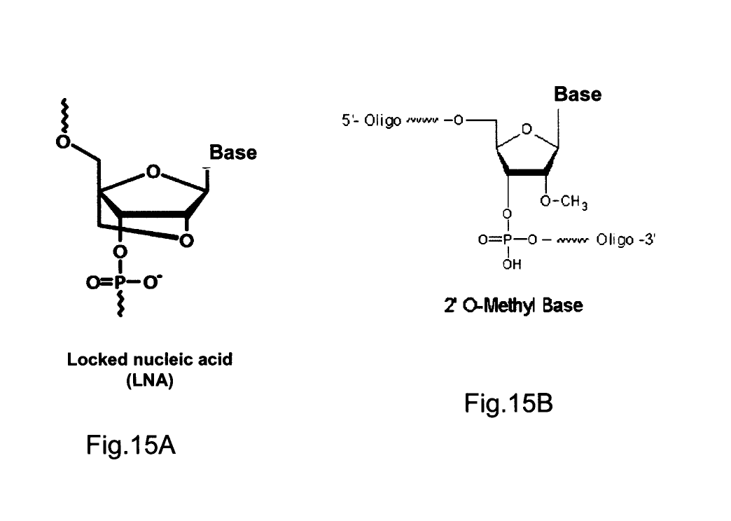

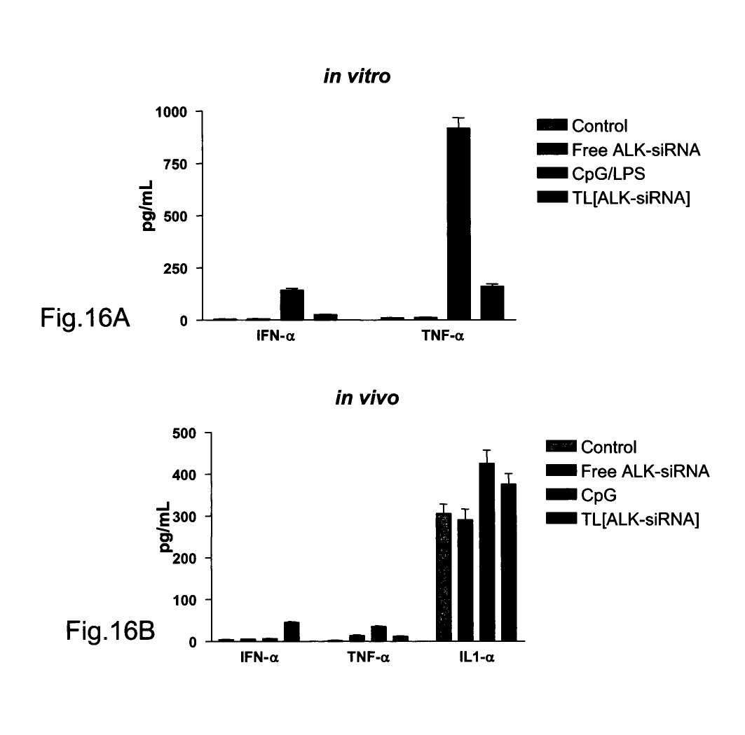

(E) The ratio of phosphorylated ERK1/2 (Extracellular Regulated Kinase) to total ERK1/2 was calculated by densitometric analysis of blots in three independent experiments as shown in FIG. 1D. Histograms show relative levels of ERK1/2 phosphorylation in SK-N-BE2 and SH-SY5Y cells. The statistical difference in ERK1/2 phosphorylation was calculated using Student's t test and resulted significantly reduced in cells transduced with A5 compared to controls with M5 (p<0.05).

FIG. 2. ALK-shRNA inhibits neuroblastoma tumor growth and progression in vivo.

Survival curves of SCID mice (n=7 for each treatment) orthotopically transplanted with neuroblastoma cell lines with the transduction of an inducible ALK-shRNA (Tet-on) in response to treatment with doxycycline.

(A)SK-N-BE2 TTA A5 cells were pretreated with doxycycline in vitro for 48 hours and then transplanted in mice maintained on treatment with doxycyline (Δ) or transplanted in mice and treated with doxycycline 7 days after orthotopic xenografts (▾). Cells kept under AIK ablation had a much less effective growth compared to control cells (▪) (P=0.001).

(B)(C) Doxycycline administered 7 days after implantation of SH-SY5Y TTA A5 and IMR5TTA A5 cells (▾) showed a statistically significant inhibition of tumor growth compared to untreated control mice (▪) (SH-SY5Y TTA A5: P=0.0011; IMR TTA A5: P=0.0004).

FIG. 3. Evaluation of entrapment, stability and release of siRNA liposomal formulation.

(A) siRNA entrapment in liposomes was analyzed by FACS after labeling of siRNA with FAM (FL1-H) and of liposomes with Rhodamine-PE (FL2-H).

(B) Stability over time of siRNA entrapped in liposomes, CCL[ALK-siRNA] controlled on agarose gel. Liposomes were incubated at 37° C. for different periods, then lysed with 0.1% Triton X-100 for 10 min at 37° C. and loaded (centre) in parallel with free ALK-siRNA (left side) and non-lysed liposomes, whole CCL[ALK-siRNA] (right side). After lysis of liposomes, entrapped siRNA resulted intact when compared to the corresponding amount (1 μg) of free siRNA. Ladder reference for length (10 bp and multiples).

(C) Stability over time of CCL[ALK-siRNA] in the presence of Rnase A 2 μg/ml at 37° C. controlled on agarose gel. A clear prevention of siRNA lability was obtained through entrapment in CCL (centre) with a very weak leakage (right side) compared to free siRNA (left side), which were already degraded after 30 min treatment with Rnase A.

(D) (E) siRNA release from liposomes was measured by dialysis of liposomes labelled with Rhodamine-PE containing siRNA labelled with FAM at 4° C. and 37° C. After one week, siRNA loss from liposomes is marginal in both conditions.

FIG. 4. ALK siRNA-mediated silencing and induction of apoptosis in neuroblastoma cell lines.

(A) (B) Effects of ALK knock-down evaluated by RT-qPCR in real time 48 hours after SH-SYSY cell transfection. Marked ALK knock-down is obtained by transfection in vitro with siRNA directed against ALK and lipofectamine RNAi MAX (FIG. 4A) or with TL[ALK-siRNA] (FIG. 4B), showing the high specificity of sequence of siRNA, that do not affect the other examined genes. The analysis of relative gene expression is reported on the y-axis as percentage of expression compared to negative, scrambled (scr) control sequences. Values are mean±95% CI of N=3 independent experiments performed in duplicate.

(C) Western blot showing an extremely selective effect of ALK protein ablation obtained after transfection with TL[ALK-siRNA] in two GD₂-positive neuroblastoma cell lines (SH-SY5Y and HTLA-230) but not in GD₂-negative cells (LB24Dagi). Both native cell lines (control) and cells treated with TL[scr-siRNA] were loaded as negative controls.

(D) (E) TL[ALK-siRNA] promotes selectively apoptosis in GD₂-positive cells (FIG. 4D) but not in GD₂-negative cells (E). Positive cells for Annexin V after treatment with TL[ALK-siRNA] were reported on the y-axis as percentage compared to control and cells treated with TL[scr-siRNA]. Values are mean±95% CI of N=3 independent experiments performed in quadruplicate. ***: P<0.001 vs control.

FIG. 5. ALK gene silencing through TL[ALK-siRNA] induces apoptosis and inhibits angiogenesis in vivo.

Immunohistochemical (IHC) and immunofluorescence (IF) stainings performed on tumor sections fixed in formaline and embedded in paraffin of SH-SY5Y subcutaneous (s.c.) xenografts treated with TL[ALK-siRNA] and compared with xenografts either untreated (control) or treated with free ALK-siRNA and TL[scr-siRNA].

Black bar: 100 μm. White bar in B, C: 50 μm; in D, E: 100 μm.

(A) IHC staining for ALK on xenografts of neuroblastoma shows complete ablation of ALK protein through treatment with TL[ALK-siRNA] and a partial decrease in expression (down-modulation) through free ALK-siRNA, likely localized around the injection sites (see arrows).

(B) IF staining for Ki67 (cell proliferation indicator) shows a considerable reduction of cell proliferation in tumors treated with TL[ALK-siRNA].

(C) TUNEL staining shows a strong induction of apoptosis through treatment with TL[ALK-siRNA].

(D) (E) Staining of endothelial cells (CD34) and perivascular cells (SMA), respectively, decreased significantly after ALK silencing through TL[ALK-siRNA] in xenografted tumor reveals a strong anti-angiogenic effect.

FIG. 5b is illustrates the percentage of positive cells for each specific marker that is reported on the y-axis of graphs as the mean±CI of randomly selected fields every 3 sections of different tumor areas. *: P<0.05, **: P<0.01, ***: P<0.001.

FIG. 6. Effect of ALK silencing through TL[ALK-siNA] on angiogenesis in neuroblastoma in vivo.

IHC performed on tumor sections as reported in FIG. 5. Black bar: 100 μm.

(A) (B) IHC staining for MMP-2 and MMP-9 shows a highly significant inhibition of the expression of both metalloproteinases of the extracellular matrix in tumors derived from s.c. xenograft of neuroblastoma cells in mice treated with TL[ALK-siRNA]vs control, tumors treated with TL[scr-siRNA] and with free ALR-siRNA.

Similar inhibitory effects through treatment with TL[ALR-siRNA] observed for expression of VEGF (Vascular Endothelium Growth Factor) (C) and its receptor VEGFR-2 (D).

In FIG. 6b is, the percentage of positive cells for each specific marker is reported on the y-axis as the mean±95% CI of 9 randomly selected fields every 3 sections of different tumor areas. *: P<0.05, **:P<0.01, ***:P<0.001.

FIG. 7. Inhibition of tumor growth in vivo through TL [ALR-siRNA]

Survival curves of mice with pseudometastatic neuroblastoma in response to treatment with siRNA liposomal formulations. Nu/nu (n=7) (A) and SCID/bg (n=8) (B) mice were treated twice a week five times with TL[siRNA] formulations 24 hours after i.v. inoculation of HTLA-230 cells.

(A) Treatment with TL[ALR-siRNA] (A) showed a significantly increased life span compared to control (▪) and to mice treated with TL[scr-siRNA] (Δ) (P=0.0002 and P=0.012, respectively).

(B) TL[ALR-siRNA] increased life span significantly (▾) compared to mice treated with other formulations, i.e. P=0.0016 vs mice treated with TL[scr-siRNA] (Δ).

FIG. 8. Studies on acute and chronic renal and hepatic toxicity (FIGS. 8A and 8B).

AST: serum glutamic oxaloacetic transaminase; ALT: glutamic-pyruvic transaminase; γGT: gamma-glutamyl transpeptidase; CRE: creatinine; BUN: blood urea nitrogen.

FIG. 9. Survival curves in SCID mice (n=7 for each treatment) undergoing subcutaneous transplantation with SK-N-BE2 TTA A5 with the transduction of an inducible Tet-on ALK-shRNA in response to treatment with doxycycline. Mice were treated with doxycycline at t₀(Δ) or 10 days after inoculation (▾). In both cases, ALK silencing produced a significantly increased life span (P=0.0008 at tₒ and P=0.0054 at day 10) compared to control mice (▪).

FIG. 10. FIG. 10A illustrates schematically the cationic liposome coated with neutral lipids (CCLs) targeted with the recognizing molecule and called TL[siRNA]. The diameter and zeta potential (FIGS. 10B and 10C) of targeted and non-targeted liposomes (CCL[siRNA]) entrapping siRNA, prepared according to the protocol described further on, resulted identical after 1 month at 4° C. in PBS (Phosphate Buffer Saline), showing a good stability of the liposomal formulation subject of the present invention.

FIG. 11. TL[scr-siRNA] binding to cells positive for GD₂ (SH-SY5Y) (FIG. 11A) and cells negative for GD₂ (FIG. 11B) was demonstrated by measuring phospholipid (PL) uptake of the preparation of liposomes labeled with [³H]-CHE. A significant dose-dependent increase in the efficiency of cellular association was observed in GD₂-positive cells treated with TL[sci-siRNA] at 4° C. (∘) and at 37° C. (•), while the same liposomal preparation without anti-GD₂ membrane antibody (antiGD₂ Mab), CCL[scr-siRNA], showed an extremely low phospholipid uptake at 37° C. (▪). Pretreatment of cells with free anti-GD₂ Mab has annulled completely the uptake of phospholipids by cells (*). Evaluation of siRNA uptake in SH-SY5Y cells was evaluated by confocal microscopy. While almost no uptake of non-targeted liposomes, CCL[scr-siRNA-FAM], was observed (FIG. 11C), GD₂-targeted liposomes, TL[scr-siRNA-FAM] were efficiently internalized (FIG. 11D). (green: TL[scr-siRNA-FAM]; red: aCD56 labeled with TRITC highlighting NCAM cell surface molecule (Neural Cell Adhesion Molecule); orange: color resulting from overlapping of green (siRNA-containing liposomes) and red (cell surface) showing TL[scr-siRNA-FAM] binding to the cell surface; blue: DAPI nuclear staining. White bar: 50 μm.

FIG. 12. Pharmacokinetics evaluated by fluorimetric analysis, expressed as percentage of administered dose of lipids (liposomes) or of remaining siRNA in blood. The formulations used are the following: free scrambled-siRNA labeled with Cy₃, identified as Cy₃-scr-siRNA (▪), liposomes labeled with Rhodamine-PE containing scrambled-siRNA, identified as Rhoda-PE-TL[scr-siRNA] (▴), or liposomes containing scrambled-siRNA, labeled with Cy₃, identified as TL[Cy₃-scr-siRNA] (▾). Liposomal formulations have long circulation time and slow elimination rates (about 10% of residual lipid vector and drug in blood 24 hours after administration).

FIG. 13. Efficiency of gene-specific silencing was evaluated by FACS analysis. SH-SY5Y cells transducing eGFP (enhanced green fluorescent protein) were treated with liposomes targeted with anti-GD₂ antibodies, labeled with Rhodamine-PE and containing siRNA directed against eGFP, identified as TL[eGFP-siRNA]or liposomes with anti-GD₂ antibodies containing control siRNA, identified as TL[scr-siRNA]. While cells treated with TL[scr-siRNA] maintain a high level of eGFP expression at 48 hours from treatment (FIG. 13A), cells treated with TL[eGFP-siRNA], after 24 hours (FIG. 13B) and 48 hours (FIG. 13C), led to reduced eGFP expression, respectively in about 10% and 30% of vital cells. eGFP expression was evaluated by FACS on FL1-H channel, Rhodamine-PE on FL2-H channel.

FIG. 14. Efficiency and specificity of gene silencing over time evaluated in SH-SY5Y through RT-qPCR. siRNA-2 sequence was chosen for this study. Native cells (not treated) were used as control.

FIG. 15. Chemical modifications in nucleotides such as LNA (Locked Nucleic Acid) or 2′-O-Methyl-ribonucleotides.

FIG. 16. TL[ALK-siRNA] immunostimulating activity was evaluated in terms of possible induction of IFN-α and TNF-α in human PBMC (peripheral blood mononuclear cells) supernatant and of possible induction of IFN-α, TNF-α, and IL1-α in mouse serum. Both TL[ALK-siRNA] and free ALK-siRNA at the administered doses induced very mild production of the analyzed cytokines, in vitro (FIG. 16A) and in vivo (FIG. 16B), compared to stimulations induced by sequences of TLR9 agonist CpG or TLR4 agonist lipopolysaccharides (LPS).

The characteristics and the embodiments of the present invention, to be intended as non-limiting examples, are described below.

Unless otherwise indicated, the execution of the described experiments involves the use of conventional chemistry, biochemistry, molecular biology, microbiology, recombinant DNA and immunology techniques that can be performed by people skilled in the art. These techniques have also been described in the literature.

The subject of this invention has been tested in vitro and in vivo, before its possible use in humans.

The experiment described below shows that ALK is an efficient therapeutic target in neuroblastoma, irrespective of its mutational status.

RNA interference or RNAi experiments were performed in a panel of neuroblastoma cell lines presenting an over-expression of both wild-type (WT) and mutated ALK gene.

Different neuroblastoma cell lines were transduced with a lentiviral system of Tet-on inducible expression (activated by doxycycline) of ALK-shRNA (TTA A5), allowing potent knock-down of ALK expression, as previously described in cells of large cell anaplastic lymphoma (Ambrogio et al., 2009; Chiarle et al., 2008; Piva et al., 2006a).

Lentiviral particles transfer the plasmid coding for shRNA (short hairpin RNA) into the target cell.

After 72 hours of treatment with doxycycline, ALK expression in two neuroblastoma cell lines, SK-N-BE2 (with wild-type ALK genotype) and SH-SY5Y (with ALK genotype presenting the point mutation that leads to F1174L amino acid substitution) transduced with shRNA (A5) specific for ALK, had considerably decreased by more than 90% compared to cell lines either non-induced or expressing a control shRNA (M5), as illustrated in FIG. 1A.

Then, it has been checked whether ALK knock-down interfered with neuroblastoma cell growth in co-culture experiments.

Cells transduced with lentivirus that express GFP (Green Fluorescent Protein) as reporter protein were mixed at 1:1 ratio with non-transduced parental cell lines.

Relative cell growth of the two populations was then measured over time with flow cytometry.

Both cell lines expressing shRNA specific for ALK (A5) showed a clear decrease in cell growth when compared to cells expressing control sh-RNA (M5), thus indicating that ALK is necessary to support neuroblastoma cell growth (FIG. 1B).

It was also evaluated whether ALK was necessary to maintain neuroblastoma cell vitality.

Therefore, neuroblastoma cell lines with wild-type ALK (SK-N-B2) and cells with mutated ALK (SH-SY5Y) transduced with shRNA specific for ALK (A5), i.e. SK-N-B2 TTA A5 and SH-SY5Y TTA A5, showed a significant and dramatic increment of apoptotic cell percentage only when A5-shRNA was induced by doxycycline, but not the M5-shRNA control (FIG. 1C).

In agreement with the reduction of cell proliferation after ALK knock-down, both SK-N-B2 and SH-SY5Y transduced cell lines showed a significant reduction of ERK1/2 phosphorylation (Extracellular Regulated Kinase 1 and 2, cytosolic proteins belonging to MAP kinase family), as demonstrated by Western blotting (FIGS. 1D, 1E), while no decrease in phosphorylation was observed in STATS or AKT (FIG. 1D), two of the main proteins involved in signal transduction that regulate cell survival in lymphomas (Piva et al., 2006a).

It has therefore been confirmed that ALK-shRNA inhibits cell growth and induces cell death in human neuroblastoma cell lines.

In addition, ALK-shRNA inhibits neuroblastoma cell growth in vivo and progression in orthotopic models.

As illustrated in FIG. 2, the so-called proof of concept in vivo was performed, i.e. the experimental test to show whether the block of shRNA-mediated ALK expression interferes with neuroblastoma cell growth also in vivo through the use of different strategies in two neuroblastoma animal models.

As first approach, SK-N-BE2 TTA A5 (ALK wt) neuroblastoma cell line, i.e. the cell line with wild-type ALK transduced with lentivirus carrying A5-shRNA specific for ALK, was treated in vitro with doxycycline for 48 hours to knock-down ALK expression and then injected in the adrenal gland of SCID mice (Severe Combined Immuno Deficiency, mice with mixed severe immunodeficiency).

Mice were treated continuously with doxycycline and cell growth was monitored over time.

SK-N-BE2 TTA A5 cells induced by treatment with doxycycline grew less efficiently compared to the same cells not treated with doxycycline (control) (FIG. 2A).

In an alternative approach, mice were treated with doxycycline from day 7 after orthotopic xenograft on. Again, ALK knock-down hindered heavily neuroblastoma tumor growth (FIG. 2A). For this reason, this approach was chosen for subsequent experiments with SH-SY5Y TTA A5 (i.e. with neuroblastoma cell lines with ALK mutated for F1174L) and IMR-5 TTA A5 (i.e. with neuroblastoma cell lines with wild-type and amplified ALK) that express A5-shRNA specific for ALK, through cell orthotopic implantation.

Tumor growth was extremely reduced when compared to non-treated control mice (FIGS. 2 B, C). As a consequence, a significant increment of life expectancy was observed in mice with subcutaneous (s.c.) xenograft of SK-N-BE2 TTA A5, both when ALK knock-down was obtained immediately after injection of cells and when the tumor mass was already stabilized (10 days) (FIG. 9).

Therefore all this shows that ALK-shRNA inhibits neuroblastoma tumor growth in vivo and progression in orthotopic models.

According to the present invention, to increase significantly dose, half-life and stability of siRNA (small interfering RNA) and therefore to allow a greater opportunity for multiple molecules to reach tumor sites and thus extend clinical use, the present invention provides the use of lipid vectors, in particular cationic liposomes coated with neutral lipids (Coated Cationic Liposomes or CCLs) that previously proved to be efficient in condensing and transporting nucleic acids or antisense oligonucleotides for release in target cells (Pagnan et al., 2000; Pastorino et al., 2001).

Since plasmid-cationic lipid lipopolyplexes or plasmid-cationic polymer lipopolyplexes have a short half-life in the circulation after intravenous i.v. injection, it was chosen to use CCLs as lipid vector for siRNA, as this type of liposomes are characterized by an increment of half-life and a reduced toxicity (Brignole et al., 2003; Pastorino et al., 2007; Stuart et al., 2000).

CCL liposomes are typically 100-200 nm in diameter (polydispersity 0.0850.15), mean 135-140 nm, with entrapment efficiency -90%.

In fact, loading capacity is approximately 10 nmol siRNA/μmol of total lipids which is comparable, though higher, to the value obtained until today by others.

The mean diameter of siRNA-containing liposomes called TL[siRNA] after coupling with anti-GD₂ antibodies (GD₂-targeted liposomes entrapping siRNA) increases by 10-30 nm (polydispersity 0.1±0.025) with a mean coupling efficiency of 50±15 μg of whole antibody/μmol of phospholipids (PL) or of 30±15 μg of antibody reduced to Fab′ fragments/μmol of phospholipids (PL).

Size and surface charge of nanoparticles was measured as a time function.

Both diameter measures and zeta potential remained identical after 1 month at 4° C. in PBS (Phosphate Buffer Saline), thus indicating a good stability of the liposomal formulation of the present invention (FIGS. 10B, 10C).

siRNA entrapment in liposomes was evaluated by labeling siRNA with FAM and liposomes with Rhodamine-PE, analyzing them with FACS.

FIG. 3A shows that about 94% of siRNA is entrapped in CCL liposomes.

To test the lability of siRNAs, which can result in a rapid degradation by serum nucleases, ALK-siRNA liposomal formulations, both treated and not treated with RNase A, were incubated at 37° C. for different durations and controlled on agarose gel 2.2%. Liposome lysis with Triton X-100 0.1% for 10 minutes at 37° C. showed that encapsulated ALK-siRNA were intact.

Conversely, whole liposomes were retained in gel wells, while only one weak band of about 20 base pairs was identified, indicating a very moderate dispersion of siRNAs outside the lipid vector (FIG. 3b).

FIG. 3C shows the effects of treatment with RNase on free ALK-siRNA and CCL[ALK-siRNA], i.e. ALK-siRNAs contained in liposomes which are the subject of the present invention, incubated at 37° C.

A clear prevention of siRNA lability is obtained when siRNA were entrapped in CCL when compared to free ALK-siRNA, which result already degraded after 30 minutes of treatment with RNase.

The stability of liposomes entrapping siRNAs was measured by dialysing liposomes labeled with Rhodamine-PE, containing siRNAs labeled with FAM at 4° C. and at 37° C.

The content of the dialysis bag was sampled at intervals and residual fluorescence of liposomes was measured with FACS analysis. In this experiment, siRNA leakage of liposomes was marginal, no more than 10-20% after 1 week both at 4° C. and at 37° C. (FIGS. 3 d, e).

siRNA-containing liposomes were prepared and directed against GD₂-positive cells called TL[siRNA].

More specifically, the following were prepared:
TL[ALK-siRNA] for siRNAs directed against ALK
TL[scr-siRNA] for control siRNAs
TL[GAPDH-siRNA] for siRNAs against GADPH (glyceraldehyde-3-phosphate dehydrogenase)
TL[eGFP-siRNA] for siRNAs against eGFP

Binding of TL[scr-siRNA] to GD₂-positive cells of SH-SY5Y neuroblastoma cell line and to GD₂-negative cells of LB24Dagi melanoma cell line was studied through measurement of the uptake of phospholipids (PL) of liposomes.

Phospholipid uptake by neuroblastoma cells increased with phospholipid concentration, showing subsaturation at 400-600 nmol PL/mL at 4° C., which is less evident at 37° C. (FIG. 11A).

The significant increase in the efficiency of cellular association observed when the temperature was increased from 4° C. (a non permissive condition for receptor-mediated endocytosis) to 37° C. is a strong indication that targeted particles are actively internalized by GD₂-positive tumor cells.

The same liposomal preparation without anti-GD₂ monoclonal antibody (mAb), defined as CCL[scr-siRNA], showed almost no PL uptake both at 4° C. and at 37° C.

Cellular preincubation for 30 minutes with soluble anti-GD₂ blocks PL uptake by cells almost completely, suggesting that TL binding [scr-siRNA] to cells occurs through a specific antigen-antibody recognition.

Conversely, GD₂-negative cells showed an extremely low uptake of phospholipids in all examined cases (FIG. 11B).

siRNA uptake was finally evaluated by confocal microscopy analysis of GD₂-positive cells, SH-SY5Y, treated with CCL[scr-siRNA-FAM], i.e. liposomes lacking anti-GD₂ antibody (not targeted) and containing control siRNAs labeled with FAM, and TL[scr-siRNA-FAM], i.e. liposomes with anti-GD₂ antibody (GD₂-targeted) containing control siRNAs labeled with FAM.

No uptake of CCL[scr-siRNA-FAM] was observed (no green signal) (FIG. 11C, left side), whereas TL[scr-siRNA-FAM] (green) were actually internalized (FIG. 11C, right side).

In general, the results obtained from the experiments described above in terms of mean dimension, efficiency of encapsulation, stability and selective targeting of TL[siRNA] show that the lipid vectors of this invention have chemicophysical characteristics meeting the requisites that a targeted non-viral particle aimed at systemic gene silencing for cancer treatment must have.

The targeted liposomal formulation according to the present invention has a good stability and long circulation time.

Long circulation time is required for small liposomes to gain access to tumor sites (Pastorino et al., 2003b).

This formulation has slow elimination time, still with approximately 10% of lipid vector and residual siRNA in blood at 24 hours from administration.

The fact that siRNA:lipids ratio changes moderately (similar slope of curves in FIG. 12) indicates a slow drug release rate.

In order to quantify the destiny of both liposome and the “drug contained therein”, pharmacokinetic studies were performed, as illustrated in FIG. 12.

In detail, pharmacokinetics of the following:
Rhodamine-PE-TL[scr-siRNA]. i.e. GD₂-targeted liposomes labeled with Rhodamine-PE and containing scrambled-siRNA control sequences,
TL[Cy₃-scr-siRNA] i.e. GD₂-targeted liposomes containing control siRNAs (scrambled RNA) labeled with Cy₃ (fluorofore emitting a wavelength in the green field),
Free Cy₃-scr-siRNA, i.e. control siRNAs (scrambled RNAs) labelled with Cy₃, free (not contained in the lipid vector),

have been evaluated and expressed as percentage of administered dose of lipids (liposomes) or residual siRNAs in blood (FIG. 12).

The proof-of-concept of the efficiency of gene-specific silencing of siRNA transport and release, subject of the present invention, was initially evaluated by FACS analysis of SH-SY5Y transducing eGFP (enhanced Green Fluorescent Protein) treated or not with TL[eGFP-siRNA].

FIG. 13 shows that about 10% of vital cells present eGFP knock-down after 24 hours (FIG. 13b) and about 30% after 48 hours (FIG. 13C) of treatment with TL[eGFP-siRNA] liposome compared to cells treated with TL[scr-siRNA], which maintained high eGFP expression up to 48 hours (FIG. 13A).

Among the three ALK-specific sequences of siRNAs tested by qRT-PCR (FIG. 14), the most effective sequence, which was therefore chosen for this study, was the following: SilencerSelect® predesigned siRNA of Ambion-Applied Biosystems (siRNA-2: ID #: s1271) sense sequence 5′->3′CAAACCAGUUAAUCCAGAAtt; antisense sequence 5′->3′ UUCUGGAUUAACUGGUUUGta. This sequence can present chemical modifications of nucleotides such as LNA (Locked Nucleic Acid) (FIG. 15A), characterized by a methylene bridge between 2′-O atoms (oxygen) and 4′-C (carbon), or 2′-O-methyl-ribonucleotides, that present hydroxyl group (OH) hydrogen substitution at position 2′ with a methyl group (CH3) (FIG. 15B), increase the stability of hybrids with mRNA (or DNA), increase the stability of enzymatic degradation by cellular nucleases and, in particular, 2′-O-methyl-ribonucleotides that make these sequences non-immunogenic or any future chemical or physical modifications implementing their specificity and efficacy and avoiding induction of immune response.

In order to test the feasibility of the use of liposomes with GD₂ target (defined as GD₂-targeted liposomes) to obtain specific and selective silencing in vitro, GD₂-positive neuroblastoma cells were transfected both with ALK-siRNA delivered by RNAiMAX lipofectamine (molecules composed of a polycationic tail to which the nucleic acid binds and of a lipid portion facilitating the passage of the nucleic acid-lipofectamine complex through the cell membrane) and with TL[ALK-siRNA].

The efficiency of ALK-specific silencing was quantitatively assessed by qRT-PCR 48 hours after transfection, clearly showing that the liposomal formulation of the present invention (FIG. 4B) leads to a marked sequence-specific decrease (down-modulation) in ALK expression comparable to conventional transfection agents in vitro for RNAi (FIG. 4A).

Neither the expression of GAPDH nor the expression of genes linked to neuroblastoma as PHOX2A/B (Longo et al., 2007) or to ALK as STAT3 (Chiarle et al., 2008) was altered by treatment with ALK-siRNA.

Western blotting analysis confirms that TL[ALK-siRNA] causes effective knock-down of ALK protein, which is highly selective for GD₂-positive neuroblastoma cells (FIG. 4C).

In addition, TL[ALK-siRNA] induces a significant increase in apoptosis of GD₂-positive cells but not of GD₂-negative cells (FIG. 4D-E), comparable to proteasome inhibitor, Bortezomib, used as positive control (Brignole et al., 2006).

This confirms the high selectivity of TL[siRNA] release system, which is the subject of the present invention, since GD₂-negative cells, LB24Dagi, do not entrap GD₂-targeted liposomes (FIG. 11B).

Considering that recent studies (Nguyen et al., 2009; Whitehead et al., 2009) have raised the question whether siRNA antitumoral effect was due to specific knock-down of target mRNA or merely to siRNA-mediated activation of the innate immune response, as illustrated in FIGS. 16A and 16B, immunostimulating activity of TL[ALK-siRNA] was tested through measurement of their possible induction of IFN-α (interferon α molecule of the immune system) and TNF-α (Tumor Necrosis Factor) in human PBMC and of IFN-α, TNF-α, IL1-α (interleukin 1-α) in mouse serum.

Both liposomal ALK-siRNA and free ALK-siRNA, at the doses used in these experiments, induced a marginal production of all cytokines in vitro and in vivo, when compared to CpG or LPS stimulations (FIG. 16A, 16B).

According to the present invention, GD₂-targeted liposomes containing ALK-siRNA cause ALK gene silencing and induction of apoptosis in vivo.

In order to evaluate TL[ALK-siRNA] potential for in vivo applications, chemical analyses were carried out on mouse serum with intravenous i.v. administration of siRNA, either free or contained in TL[siRNA] targeted liposomes.

TL[siRNA] is well tolerated without clear toxicity (no weight loss nor skin rash) or acute and chronic toxicity of liver and kidney (FIGS. 8A and B).

In order to confirm ALK-specific silencing and ascertain the impact of TL[ALK-siRNA] treatment on tumor cell proliferation, vitality and apoptosis in vivo, immunohistochemical and immunofluorescent staining was performed on tissue from tumors generated through subcutaneous s.c. inoculation of SH-SY5Y cells in SCID mice.

Tumors were treated with intramass injection of free ALK-siRNA or TL[ALK-siRNA] or TL[scr-siRNA] and compared to non-treated controls.

Immunohistochemical staining (IHC) for ALK showed potent knock-down of ALK protein through TL[ALK-siRNA] and a partial decrease through free ALK-siRNA, likely localized around injection sites (FIG. 5A).

Treatment of tumors with TL[ALK-siRNA] reduced the proliferation of neuroblastoma cells, as demonstrated by the significant decrease in the percentage of cells positive for Ki-67 (indicator of cell proliferation) compared to free ALK-siRNA (FIG. 5B).

It is worth noting that tumors derived from SH-SY5Y xenografts treated with TL[ALK-siRNA] show a highly significant induction of apoptosis, as ascertained with TUNEL staining (FIG. 5C).

TL[scr-siRNA] caused no effect on any marker.

Effects of TL[ALK-siRNA] treatment on angiogenesis were studied on sections of tumor derived from the same subcutaneous xenografts.

ALK silencing through TL[ALK-siRNA] led to a strong antiangiogenic activity, as indicated by a significant decrease in vessel density, as determined by staining of endothelial cells with aCD34 (FIG. 5D) and staining of perivascular cells with aSMA (FIG. 5E).

In addition, treatment with TL[ALK-siRNA] has drastically downmodulated both VEGF (Vascular Endothelium Growth Factor) expression (FIG. 6C) and VEGF-R2 receptor expression (FIG. 6D), in agreement with the antiangiogenic effect due to ALK knock-down.

Finally, in tumors treated with TL[ALK-siRNA] compared to control and to free ALK-siRNA (P<0.001), a marked reduction of the expression of MMP-2 (FIG. 6A) and MMP-9 (FIG. 6B), markers of tumor invasion, was observed.

The whole of the immunohistological results obtained clearly indicate an essential role of TL[ALK-siRNA] in inhibiting tumor angiogenesis in vivo.

Finally, mice with pseudometastatic neuroblastoma, obtained by intravenous inoculation of HTLA-230 (neuroblastoma cell line carrying wild-type ALK), were treated with different siRNA formulations hours after inoculation of neuroblastoma cells, twice a week for 5 times. As shown in FIG. 7A, mice treated with TL[ALK-siRNA] liposomes showed a significant increment of survival compared to control mice or to mice treated with TL[scr-siRNA].

After 150 days from implantation of cells, about 40% of mice treated with TL[ALK-siRNA] were still disease-free whereas all mice treated with free siRNA or TL[scr-siRNA] had died of metastatic disease.

Previous preclinical studies showed that lysis of neuroblastoma cells through anti-GD₂ antibody depended on antibody-dependent and complement-dependent cellular cytotoxicity (Pastorino et al., 2003c; Raffaghello et al., 2003a; Yu et al., 1998).

Therefore, intravenous i.v. inoculation of HTLA-230 in mice with more severe immunodeficiency (SCID/bg) (mice lacking NK cells besides B and T cells) was repeated and a significant increase in survival of mice treated with TL[ALK-siRNA] was obtained compared to mice treated with other formulations (FIG. 7B) and thus excluding the role of antibody-mediated antitumoral response in the used animal models.

Similar results were obtained in mice inoculated with LAN-5 neuroblastoma line (with ALK mutated for R1275Q) (data not shown).

In conclusion, silencing of ALK protein mediated by specific siRNA-ALK entrapped in liposomes targeted with aGD2 specifically for neuroblastoma cells both in vitro and in xenografts in vivo was demonstrated to have therapeutic efficacy.

In fact, neuroblastoma cell lines which transport wild-type, mutated or amplified ALK expressing stably shRNA, with scrambled sequences or directed against ALK, were engineered with the aim to verify the effect of ALK ablation on cell signal transmission pathways, cell proliferation and apoptosis in vitro, as well as on growth and tumorigenesis in vivo in murine models with heterotransplantation.

Current data recommend orthotopic implantation of neuroblastoma tumor cells in animal recipients for studies on tumor progression, angiogenesis, invasion, and metastasis (Chambers et al., 2002).

The biologically and clinically relevant orthotopic model which best reflects advanced neuroblastoma growth in children (Pastorino et al., 2003a; Pastorino et al., 2009) was therefore chosen, and it was possible to obtain the proof of concept, a confirmation of validity, and a realistic vision of clinical potential and of ALK targeting in mice implanted with engineered neuroblastoma cell lines expressing stably ALK-shRNA.

Of great interest is ALK knock-down in orthotopic and subcutaneous neuroblastoma models, which led to an important inhibition of tumor growth, irrespective of ALK mutational status.

The pseudometastatic models described above were chosen for the evaluation of the therapeutic efficacy of siRNA directed against ALK, since neuroblastoma cells injected intravenously in mice mimic the metastatic degree observed in patients with advanced stage neuroblastoma (Pastorino et al., 2003c; Raffaghello et al., 2003a).

Neuroblastoma cells circulating in blood and micrometastases in the bone marrow at the moment of the first surgical intervention in patients with neuroblastoma are strong predictors of relapse and, since they express a high level of the cell surface receptor disialoganglioside GD2 (Handgretinger et al., 1992; Mujoo et al., 1987), they are actually a good target for release systems through nanoparticles.

In addition, since bone marrow micrometastases are a direct measure of systemic spreading ability of tumor cells, the establishment of a model mimicking the clinical situation allows a more realistic evaluation of antitumor therapies.

The implemented plan of treatment, started 24 hours after neuroblastoma cell injection, has been deliberately chosen to allow the evaluation of the effects of siRNAs encapsulated in targeted liposomes during the metastatic cascade, i.e. during the stages in which tumor cells are in the intravascular circulation and bind the endothelium or when extravasation, invasion and stromal colonization occur (Pastorino et al., 2003c, Raffaghello et al., 2003b).

A new nanoparticle or lipid vector has therefore been developed for selective release in vivo of a high level of siRNA directed against an oncogene, in particular against ALL to tumor cells expressing GD₂ on surface, in particular neuroectodermal tumor cells, i.e. neuroblastoma cells.

In fact, siRNA-containing liposomes can be specifically targeted, i.e. can be provided with antibodies against surface proteins of tumor cells and therefore the possibility of targeting specific cells and internalizing lipid nanovectors is essential to kill tumor cells effectively.

These characteristics are extremely important when dealing with tumor cells that are difficult to transfect, such as neuroblastoma cells, and also the internalization of nucleic acids can be a limiting step in this process (Corrias et al., 1997; Giles et al., 1998).

Thanks to the present invention, i.e. thanks to nanoparticles or lipid vectors entrapping gene-specific siRNA having tumor cells as target thanks to the presence on their surface of specific anti-GD₂ antibodies, effective knock-down of oncogenes, in particular ALK, is possible, together with a potent inhibition of tumor cell proliferation, induction of apoptosis, and a highly significant inhibition of tumor growth.

Packaged siRNAs in the liposomal formulation which is the subject of the present invention are stable, resistant to degradation by extrinsic RNases and do not leak from nanoparticles.

The presence of the lipid vector directed against tumor cells expressing tumor-specific proteins on their surface protects siRNAs against degradation by serum nucleases, increases their membrane permeability with consequent increase in cellular uptake, improves their pharmacokinetics, and makes intracellular release more efficient, avoiding non-specific distribution.

ALK-siRNA liposomal formulation which is the subject of the present invention is effective in cells expressing wild-type, amplified or mutated ALK.

Different action mechanisms seem to be involved in siRNA-mediated ALK knock-down, able to block neuroblastoma cell proliferation and to induce increased necrosis/apoptosis.

It has been reported that ALK inhibition through small inhibitors leads to decreased activation of ERK1/2, AKT, and STAT3 pathways in neuroblastoma cell lines, and these proteins have been indicated as ALK downstream targets (Janoueix-Lerosey et al., 2008; Passoni et al., 2009).

According to the present invention, the ablation of ALK expression through the use of more selective systems, i.e. through molecules mediating specific RNA interference or RNAi, leads to significant inhibition of MAPK (MAP kinase) pathway, as shown by decreased phosphorylation of ERK 1/2 that are likely the most relevant proteins associated with this molecular process in neuroblastoma.

In agreement with the above, neuroblastoma cells should likely be sensitive to the AKT inhibitor perifosine through an ALK-independent action mechanism, as indicated by its efficacy in cells carrying both mutated and wild-type ALK (Li et al., 2010).

In addition, the TL[ALK-siRNA] liposomal formulation of this invention exerted its higher ability in inhibiting angiogenesis and tumor invasion compared to siRNAs administered in their free form.

Many recent studies involve angiogenesis as an essential mechanism regulating neuroblastoma growth and anti-angiogenic therapies are becoming extremely innovative (Rossel et al., 2008).

Sudden growth of new blood vessels from preexisting capillaries under the influence of the expression of an angiogenic growth factor such as VEGF and its receptor VEGFR-2 is well known (Ribatti and Ponzoni, 2005; Ribatti et al., 2002).

Even HIF-1α/VEGF signal pathway regulates tumor angiogenesis and invasion (Du et al., 2008).

Since hypoxia-induced decrease in the level of p53 protein (oncosuppressor acting in response to DNA damages) is known to result in resistance of cancer cells to apoptosis induced by DNA damage (Cosse et al., 2009), it is possible to suppose that HIF-1α inhibition through ALK silencing can decrease HIF-1α-mediated VEGF production, leading to apoptosis-mediated cell death and subsequent block of tumor neoangiogenesis.

The liposomal formulation which is the subject of the present invention, having neuroblastoma cells as target and containing siRNA against ALL is extremely potent in downmodulating angiogenesis-related CD34, SMA, VEGF/VEGFR-2, as well as invasiveness-related MMP-2 and MMP-9 proteins, thus confirming the antiangiogenic effect of siRNA-mediated ALK knock-down in neuroblastoma tumors.

It must be remembered that matrix metalloproteinases not only can be used as tumor markers, but are also involved in tumor growth, angiogenesis, invasion, and metastasis.

This antiangiogenic response can also be used for inhibition of secondary tumors following anti-neuroblastoma therapies (Gesundheit et al., 2007).

In conclusion, it has been demonstrated that ALK is a key therapeutic target in neuroblastoma, irrespective of its gene status.

Intravenous release of lipid nanoparticles having neuroblastoma cells as target and carrying ALK-specific siRNAs as single agents, in neuroblastoma mice, is very well tolerated and effective.

All this confirms that it is possible to develop anticancer strategies using therapies based on siRNAs as adjuvant therapy for advanced stage neuroblastoma or for disease resulting from incomplete surgical removal or early metastatic lesions.

Even though, in the present invention, the efficacy of the use of vectors containing sequences specific for ALK gene against GD₂-positive neuroblastoma cells has been extensively described, other neuroectodermal tumors as melanoma and neurosarcoma and other human additional tumors expressing abundant quantities of the GD₂ epitope can be treated in a similar way, thus benefiting from this new and potent therapeutic strategy based on the liposomal formulation of gene-specific siRNAs having tumor cells as specific target.

It is therefore possible to develop similar lipid vectors transporting molecules that mediate RNA interference or RNAi for knock-down of other oncogenes different from ALL which vectors have as target the cells expressing specific surface markers that depend on the specific tumor type, which allows therapies having specific molecules as target to be designed.

In addition, the clinical introduction of liposomal formulations of siRNAs selective for ALK can have a role in a combined treatment with small

ALK-inhibiting molecules and/or chemotherapeutic agents.

Therefore, the present invention relates also to a pharmaceutical composition comprising one or more of these vectors which pharmaceutical composition, alone or in combination with other types of vectors or chemical and/or biological agents, either free or entrapped, can have a clinical application for the treatment of pediatric tumors.

### EXPERIMENTAL PROCEDURES

### Cell Lines and Cultures

In order to cover extensively the different phenotypes shown by neuroblastoma cells, five human cell lines expressing GD₂, in particular HTLA-230 (wild-type or WT ALK), IMR-5 (with ALK amplification), SK-N-BE2 (wild-type or WT ALK), SH-SY5Y (with ALK mutation for F1174L), and LAN-5 (with ALK mutation for R1275Q) were used and grown as described (Pagnan et al., 2000). Two human cell lines were chosen as GD₂-negative controls: HeLa, derived from cervical carcinoma cells, purchased from American Type Culture Collection (ATCC), and LB24Dagi, derived from melanoma cells (Istituto Dermopatico dell'Immacolata, Dept. of Molecular and Cellular Biology, Rome, Italy). SH-SY5Y cells were stably transfected with pcDNA3.1eGFP vector (University of Erlangen-Nuremberg, Erlangen, Germany). Only cells in the exponential growth phase were used during experiments. All cell lines were tested for mycoplasma contamination, evaluation of cell proliferation and morphology, after thawing and within four passages in culture.

Therefore, cell lines derive from cell banks from anonymous donors.

Sh-RNA Constructs and Transduced Cell Lines

Cells with inducible lentiviral sh-RNA were obtained with co-transduction of pLv-tTRKRAB and pLVTHM vectors (Dr. D. Trono, Lausanne, Switzerland) containing ALK-shRNA (A5) or sh-RNA control (M5) cassettes, as described (Piva et al., 2006a; Piva et al., 2006b; Taulli et al., 2005). Cells were selected on a high-speed cell sorter (MoFlo High-Performance Cell Sorter, DAKO Cytomation) to normalize fluorescence intensity and percentages of transduced cells.

ALK silencing was obtained when 1 μg/mL of doxycycline was added to the medium for at least 72 hours. The efficiency of gene silencing and the downstream effects on the other genes was evaluated at 24, 48 and 72 hours after transfection by real-time RT-qPCR and at 72 hours by Western blotting.

Reagents and Chemical Products for Liposomal Preparation

Hydrogenated Soya Phosphatidylcholine (HSCP), cholesterol (CHE),1,2-distearoyl-glycero-3-phsphoethanolamine-N-[methoxy(polyethylene glycol)-2000] (DSPE-PEG₂₀₀₀), 1,2-distearoyl-sn-glycero-3-phosphoethanolamine-N-[methoxy(polyethylene glycol)-2000] modified with a malemidic group in the distal term of the chain (1,2-distearoyl-sn-glycero-3-phosphoethanolamine-N-[maleimide(polyethylene glycol)-2000] DSPE-PEG₂₀₀₀-MAL, 1,2-dioleoyl-1-3-trimethylammonium-propane (DOTAP), and 1,2-dipalmitoyl-sn-glycero-3-phosphoethanolamine-N-(Lissamine Rhodamine B Sulfonyl) (Rhoda-PE) were purchased from Avanti Polar Lipids (Alabaster, Ala.).

Membranes in Nuclepore polycarbonate (pore size 0.4, 0.2, and 0.1 μm) were purchased from Avestin Inc. (Ottawa, ON, Canada). 2-Iminothiolane (Traut's reagent) was obtained from Sigma (Sigma Chemical Co., St. Louis, Mo.). A/G protein column, ImmunoPure IgG elution buffer were purchased from Thermo Scientific-Pierce (Rockford, Ill., USA). Bio-Rad Protein Assay reagent was purchased from Bio-Rad Laboratories (Mississauga, Ontario, Canada). Sephadex G-50, Sepharose CL-4B and scintillation fluid for aqueous count was purchased from Amersham Pharmacia Biotech (Baie d'Urfe, Quebec, Canada). Cholesteryl-[1,2-³H—(N)hexadecyl ether ([³H]CHE; 1.48-2.22 TBq/mmol) was purchased from Perkin-Elmer Biosciences (Boston, Mass.).

Hybridoma cell line 14.G2a, which secretes G2a murine immunoglobulin (IgG2a) specific for GD₂ antigen, was provided by The Scripps Institute, La Jolla, Calif.

All the other chemical products were provided with purity of analytical grade or with the highest purity available.

In Vitro Transfection of siRNAs in Neuroblastoma Cell Lines.

The conditions of in vitro transfection for gene silencing through siRNAs were optimized in 6-well plates (2×10⁵ cells) for SH-SY5Y, IMR-32, HTLA-230 cell lines using Lipofectamine RNAiMAX (Invitrogen) and SilencerSelect® GAPDH siRNA labeled with FAM (Ambion-Applied Biosystems) at different molarities. The efficiency of transfection was evaluated by fluorescence microscopy or FACS. For each candidate gene three pre-designed SilencerSelect® gene-specific siRNAs (Ambion-Applied Biosystems) in parallel with a scrambled control (Silencer®Negative Control #1, Ambion-Applied Biosystems), one positive housekeeping control (SilencerSelect® GAPDH siRNA, Ambion-Applied Biosystems), one blank with only the transfection agent and the native cells were tested. The most effective siRNA for ALK was selected (our siRNA-2 for ALK code: ID #: s1271, Ambion-Applied Biosystems) for further experiments and TL[siRNA] preparation (see further on). Transfection was set at 100 nM siRNA and performed both in adherent cells (forward transfection) in D-MEM lacking serum without antibiotics and arrested after 14 hours with the complete medium, and in cells just collected in suspension (inverse transfection), in complete D-MEM medium without antibiotics. The efficiency of gene silencing and the downstream effect on the other genes was evaluated at 24, 48, and 72 hours after transfection by RT-qPCR in real time and at 72 hours by Western blotting.

The conditions of in vitro transfection of neuroblastoma cells by TL[ALK-siRNA], TL[scr-siRNA] and TL[GAPDH-siRNA] were optimized for GD₂-positive, SH-SY5Y and HTLA-230 cells, and GD₂-negative, LB24Dagi cells. The final concentration of siRNA was set at 750 nM siRNA/750 nM PL, which is similar to the concentration of the siRNA liposomal formulation used for in vivo studies. Subsequently, experiments were performed to evaluate the knock-down in terms of mRNA reduction level after siRNA treatment of cells in 6-well plates (about 2×10⁵ cells) while the protein knock-down (about 1×10⁶ cells) on 10 mm Petri dishes. In any case, native cells as well as cells treated with TL[siRNA] were incubated at 37° C. with moderate shaking for two hours in round-bottom tubes with small volumes. The following liposomal formulations were used: TL[ALK-siRNA], TL[scr-siRNA] as negative control and TL[GAPDH-siRNA] as positive control of transfection (only for RT-qPCR) to allow the formation of cell-liposome complexes. After two hours, cells were seeded in their respective culture plates. The cells for mRNA evaluation were collected hours after treatment while cells for protein evaluation underwent a second treatment at 48 hours and were collected at 72 hours.

The efficiency of gene silencing and the downstream effects on the other genes were evaluated at 48 hours after transfection by RT-qPCT in real time and at 72 hours by Western blotting.

Preparation of TL[siRNA] (Coated Cationic Liposomes Having GD₂ as Target and Entrapping siRNA).

Reimer and colleagues demonstrated that plasmid DNA could be extracted in organic solvents when the cationic lipid was present, presumably forming inverted micelles in the organic solvent with the plasmid inside the inverted micelle and the cationic lipid acyl chains oriented towards the organic phase (Reimer et al., 1995). On the basis of this assumption, we encapsulated siRNA in small and stable CCLs, that are prepared and purified following the method by Stuart and colleagues (Stuart et al., 2000), slightly modified by us (Pagnan et al., 2000; Pastorino et al., 2001; Brignole et al., 2004) considering also the number of negative charges of double-stranded RNA molecules (21mers). In short, siRNA was complexed with cationic lipids and coated with neutral lipids: 1000 μg of siRNA in 500 μl of distilled-deionized water (ddH₂O) were mixed with 3.4 μmol of DOTAP in 500 μL of CHCl₃ and 1060 μl of MeOH to form a Bligh-Dyer monophase. After 30 minutes at room temperature, 500 μl of CHCl₃ and 500 μl of ddH₂O were added. The mixture underwent vortexing and was centrifuged at 800×g for 7 minutes at room temperature. The upper aqueous methanol layer phase was removed. In these conditions, that were developed in pilot studies using 10 μg of siRNA and different quantities of DOTAP, 90-96% of siRNA was recovered in the organic phase. The resulting negative to positive charge ratio of siRNA-lipids is 1:1. After extraction, 8.6 μmol of cholesterol and appropriate quantities of HSCP, DSPE-PEG₂₀₀₀, or DSPE-PEG₂₀₀₀-MAL were added to the organic phase to provide cholesterol to phospholipids molar ratios of 1:2. ddH₂O was added to provide a final phospholipid (PL) concentration of 20 mM. The mixture was sonicated for 5 minutes and the organic phase was removed through rotating evaporation under N₂ flow until the system returned back to the aqueous phase. Liposomes were extruded in sequence through 400 nm, 200 nm and 100 nm polycarbonate filters. Liposome size, polydispersity and zeta potential were analyzed through dynamic light scattering using fixed-angle) (90° Zeta sizer Nano-S particle counter (Malvern Instruments, UK). Possible free siRNAs were separated from liposomes by passing the liposomes over a Sephadex G-50 column pre-balanced in HEPES buffered saline solution (25 nM of HEPES, 140 nM of NaCl, pH 7.4). Finally, the anti-GD₂ monoclonal antibody was coupled with the maleimidic end of DSPE-PEG₂₀₀₀-MAL to provide this new siRNA-entrapping nanoparticle with targeting for GD₂-positive cells. The quantity of siRNA encapsulated in CCLs was evaluated by solubilizing the liposomal preparation with 40 nM of sodium deoxycholate for 1 hour 30 minutes at room temperature (RT) followed by spectrophotometry measurement at 260 nm.

In some preparations, cholesteryl-[1,2-³H—(N)hexadecyl ether ([³H]-CHE) is added as non-exchangeable and non-metabolizable lipid tracer. In some experiments, 1,2-dipalmytoyl-sn-glycero-3-phosphoethanolamine-N-(Lissamine Rhodamine Sulfonyl) (Rhoda-PE) in 1 mol % of total phospholipids (PL) is used as fluorimetric lipid tracer.

Uptake Studies and In Vitro Cell Binding

Cells were incubated for 1 hour at 4 and 37° C. (non permissive and permissive temperature, respectively, for receptor internalization) with [³H]-CHE labeled targeted liposomes, at 0.1-0.8 μmol PL/mL concentrations. After extended washing, cells were trypsinized and lysed with 1 N NaOH for protein evaluation and 8-counting. The comparison for binding to tumor cells between free antibody (mAb) and mAb coupled with liposomes (anti-GD₂) was determined by adding a 50 times excess of free mAb for 30 minutes before adding TL labeled with [3H]-CHE (Pagnan et al., 2000; Pastorino et al., 2001).

Uptake and internalization of scrambled siRNAs, labeled with FAM and encapsulated in liposomes in cells expressing GD₂ were detected by fluorescence confocal microscopy. Cells were trypsinized, counted and incubated at 37° C. at different concentrations with liposomes either not targeted or targeted with aGD₂ antibody (i.e. having GD₂ as target). After cytospin washing and centrifugation, the samples were fixed and stained with a monoclonal antibody specific for N-CAM cell adhesion molecule (a-CD56 Zymed, Invitrogen) to reveal the localization of the plasma membrane. Binding of primary antibody was detected with anti-mouse rabbit antibody conjugated with tetramethylrhodamine isothiocyanate (PE) and cell nuclei stained with 4′ 6-diamidine-2-phenylindole (DAPI). Cellular distribution of FAM (green), PE (red) and DAPI (blue) fluorescences were analyzed by TCS SP2 AOBS laser scanning spectral confocal microscopy (Leica, Heidelberg, Germany).

siRNA Stability and Release

In order to test siRNAs lability that can lead to their rapid degradation by serum nucleases, siRNA liposomal formulations were incubated at 37° C. for different times with and without RNases A (2 μg/ml) and tested on agarose gel 2.2% (Invitrogen).

Entrapment of scr-siRNAs and FAM-scr-siRNAs (10% of total) (Silencer® Negative Control #1, Ambion-Applied Biosystems) in liposomes labeled with Rhoda-PE was evaluated and analyzed using FACS (Fluorescence Activated Cell Sorter) (FACS Calibur, Beckton Dickinson, San Jose, Calif., USA) with xenon lamp. Fluorescence intensities of FAM and rhodamine (Rhoda) were recorded through FL1-H and FL2-H channel reading, respectively.

The possible release of siRNAs contained in liposomes with radioactively labeled cholesterol ([³H]-cholesterol) was measured by dialyzing siRNA-containing TL in PBS with 25% of human plasma from healthy donors or in complete medium in a large volume of the same solvent at 4° C. and at 37° C., using dialysis tubes with molecular mass limit of 100,000 daltons and the content sampled at intervals for radioactivity and siRNA measurement.

In Vitro Cell Proliferation and Co-Culture Test

Specific inhibition of neuroblastoma cell proliferation was determined with co-culture experiments in which SK-N-BE2 TTA and SH-SY5Y TTA cells transduced with A5 or with M5 were mixed at 1:1 ratio with parental cell lines non-transduced and cultured in standard conditions for 5 days. eGFP expression was checked over time by FACS and normalized against the initial time point, as described (Piva et al., 2006a).

Design of Primers and Analysis of Gene Expression by Real-Time RT-qPCR.

Total RNA was extracted from cell lines of interest using RNeasy mini and micro kit (QIAGEN, GmbH Hilden, Germany). The analysis of mRNA expression level of target genes and positive control (GAPDH) were performed by two-phase real-time RT-qPCR using a retrotranscription based on random priming (High Capacity cDNA Reverse Transcription Kit, Applied Biosystems-Ambion) and on the use of SYBR®Green I (Platinum® SYBR® Green qPCR Su-perMix-UDG, Invitrogen) according to the manufacturer's conditions.

Primers were designed using Primer 3 software (http://fokker.wi.mit.edu/primer3/input.htm (Rozen and Skaletsky, 2000).

After testing the efficiency of primer amplification through standard curve, the triplicates of each cDNA sample (12.5 ng) were amplified in iCycler (Bio-Rad Laboratories, Hercules, Calif.) through initial denaturation at 95° C. for 2 minutes, followed by 50 cycles at 95° C. for 15 seconds and at 60° C. for 30 seconds. Melting curves between 55° C. and 95° C. were also calculated, with a 0.5° C. increment every 15 seconds. PCRs were repeated at least twice.

Three human genes (POLR2A, POLR3F, NDUFB3) were chosen as endogenous controls and data were normalized using Bestkeeper software (http://www.gene-quantification.de/bestkeeper.html). For relative quantification of gene expression in native cell lines, we prepared an equimolar pool of RNA from eight normal tissues of different embryonal origin (adrenal gland, bladder, breast, brain, colon, lung, placenta, and prostate gland) (FirstChoice® Human Normal Tissue Total RNA, Ambion-Applied Biosystems) to be used as reference sample. In transfected cells, the gene expression level was compared with the appropriate negative controls (i.e. native cells, the transfecting agent alone, the scrambled sample or the empty vector).

The Ct comparative method was adopted for the relative quantification of gene expression in transfected and native cell lines and data were analyzed with qGene software (http://www.gene-quantification.de/download.html#ggene) (Muller et al., 2002) implemented to correct amplification efficiencies.

Western Blotting

Inhibition of gene expression was confirmed at protein level by Western blotting, starting from total cell lysates using specific antibodies, as previously performed (Pagnan et al., 2000; Pastorino et al., 2003b; Piva et al., 2006). For immunobloting, proteins were separated by SDS/PAGE and transferred on nitrocellulose, incubated with the specific antibody, and immunocomplexes were detected with anti-mouse Ig antibody (produced in sheep) conjugated with horseradish peroxidase (Amersham), and visualized through enhanced chemiluminescent reagent (Amersham) according to the manufacturers.

The membrane was incubated with monoclonal antibodies against ALK (1:2000; Zymed, Seattle, Wash.), phospho-tyr (PY100; 1:1000; Cell Signalling Technology, Beverly, Mass.) or polyclonal antibodies against Stat3 (1:1000), phospho-Stat3 (Y705) (1:1000), Erk1/2 (1:1000) (Santa Cruz Biotechnology, Santa Cruz, Calif.), AKT (1:1000), phospho-Erk1/2 (1:500), phospho-AKT (1:500) (Cell Signalling Technology).

In Vitro Cytotoxicity and Apoptosis after Treatment with TL[ALK-siRNA]

In vitro cytotoxicity of free siRNA or TL[ALK-siRNA] was evaluated in a panel of neuroblastoma cell lines and in a GD₂-negative cell line by counting cells stained with trypan blue vital stain. The analysis of apoptosis was performed on parallel cultures using Annexin-V-FLUOS Staining Kit (propidium iodide), Roche Diagnostics (Quebec, Canada), as previously described (Brignole et al., 2006; Marimpietri et al., 2005). Alternatively, apoptosis was measured by TMRM staining (Piva et al., 2006). Both analyses were performed in quadruplicates according to the conditions and the concentrations used for in vitro transfection of siRNA, adapted for 96-well plates. Treatments were scheduled at 0, 48 and 96 hours and evaluation was performed at 120 hours. Four additional cell replicates were treated with Bortezomib 20 nM for 24 hours to be used as positive control in analysis of apoptosis.

Animal Models

All in vivo experiments were performed with 3-8 mice per group and repeated twice. Body weight and general physical conditions of the animals were recorded daily, and mice were sacrificed for cervical dislocation after xylazine administration (Xilor 2%, Bio98 Srl, Milan, Italy) when they showed the first signs of poor health, such as abdominal dilatation, dehydration, or paraplegia.

All the animals were purchased from Harlan

Laboratories (Harlan Italy, S. Pietro al Natisone, Udine, Italy) and kept in specific pathogen-free conditions. Animal experiments were reviewed and approved by the Ethics Committee of the National Cancer Research Institute of Genova, Italy and of the Italian Ministry of Health.

As detailed further on, three xenograft models were used:

a) orthotopic model with nu/nu mice injected with SK-N-BE2 TTA A5, SH-SY5Y TTA and IMR-5 TTA A5 cell lines in the left adrenal gland (Pastorino et al., 2003a; Pastorino et al., 2008),

b) pseudometastatic model with SCID/bg and athymic (nu/nu) mice injected intravenously (i.v.) in the tail vein with HTLA-230 and LAN5 cells (Pastorino et al., 2003c; Raffaghello et al., 2003),

c) subcutaneous model with SCID mice inoculated with SK-N-BE2 TTA A5 and SH-SY5Y cells in the mediodorsal region (Pastorino et al., 2003b).

For the subcutaneous (s.c.) animal model, 20×10⁶ SH-SY5Y or SK-N-BE2 cells, native or TTA A5 (shALK-transduced), were inoculated in the mediodorsal region of five-week female SCID mice. Tumor expansion over time as well as response to treatment were measured with calipers, as described (Pastorino et al., 2003b; Pastorino et al., 2010).

For the pseudometastatic animal model, 4-week female SCID/bg and athymic (nu/nu) mice were injected intravenously (i.v.) in the caudal vein with 3.5×10⁶ HTLA-230 tumor cells, as described (Pastorino et al., 2003c; Raffaghello et al., 2003).

In the orthotopic animal model, 5-week female nu/nu mice were anesthetized with ketamine (Imalgene 1000, Merial Italia SpA, Milan, Italy), submitted to laparatomy, and 2.5×10⁶ SK-N-BE2 TTA A5 or 5×10⁶ IMR-5 TTA AS or 1.5×10⁶ SH-SY5Y TTA A5 cells were injected into the capsule of the left adrenal gland, as described (Pastorino et al., 2003a; Pastorino et al., 2008). No mice died as a result of this treatment. Mice were monitored at least twice a week for tumor growth testing and were sacrificed as mentioned above. When established, doxycycline was administered at a concentration of 1 mg/mL in drinking water to which 1% of sacharose was added to improve taste.

In Vivo Therapeutic Studies

For s.c. animal models obtained through inoculation of SK-N-BE2 TTA A5 cells, doxycycline was administered in drinking water (for total 300 mg/Kg/day) at time 0 or 10 days after cell inoculation on alternate days.

For ALK-shRNA-transfected orthotopic animal models, obtained through implantation of SK-N-BE2 TTA A5, IMR-5 TTA and SH-SY5Y TTA AS cells, tumors were allowed to grow from the implanted cells for seven days, before injection of treatment with doxycycline. In some experiments, SK-N-BE2 TTA A5 were treated in vitro with doxycycline for 48 hours before orthotopic injection.

For the pseudometastatic model obtained through inoculation of HTLA-230 and LAN-5 cells, mice were treated i.v. 24 hours after inoculation of tumor cells with free ALK-siRNA, TL[ALK-siRNA] and TL[scr-siRNA] (1.25 mg/kg siRNA) twice a week for total five times.

Control mice received weekly injections of HEPES buffered saline solution. Experiments were performed twice. Mice routinely monitored for weight loss were sacrificed when poor health signs became manifest. Survival times were used as main criterion for determining the efficacy of treatment.

Pharmacokinetic Experiments

Athymic (nu/nu) mice were inoculated through the caudal vein with a single dose of Rhoda-PE-TL[scr-siRNA], scr-siRNA labeled with Cy₃ entrapped in TL (0.8 μmol PL/mouse), and free scr-siRNA labeled with Cy₃ (Cy₃-scr-siRNA). At selected time intervals (2, 12, 24, and 48 hours) post injection, mice (3/group) were anesthetized, blood was collected through the retro-orbital sinus of each mouse into a tube containing heparin (2 IU/mL; Parke-Davis, Milan, Italy) and mice were sacrificed. Blood was centrifuged (500 g for 10 minutes at 4° C.) and Cy₃ and Rhoda-PE labels were evaluated on 100 μL of isolated plasma using a fluorescence plate reader (INFINITE M2000 Mono-chromator Instrument, TECAN, GmbH, Grodig/Salzburg, Austria). Excitation/emission of Cy₃ and Rhoda-PE: 547/563 and 557/571 nM, respectively. Pharmacokinetic data were expressed as percentage of injected dose remaining in blood at the different time moments post injection.

Histological Analysis

SH-SY5Y s.c. tumors were allowed to grow for 13 days and to reach a size of about 150 mm³ before starting intratumoral treatment with free ALK-siRNA, TL[ALK-siRNA] and TL[scr-siRNA] (50 μg siRNA/mice for three consecutive days). In all the experiments, the group of control mice received HEPES buffered saline solution. On the day following the last treatment, mice were anesthetized with xylazne and sacrificed, and tumor masses were recovered for immunohistochemical analyses (IHC). Tumors were then fixed in formalin and then embedded in paraffin. Tissue sections embedded in paraffin (3 μm thickness) were subsequently deparaffined through a xylene-ethanol sequence, rehydrated in graduate ethanol solution and in TRIS buffered saline solution (TBS, pH 7.6), and then processed for antigen recovery bringing tissue sections to ebullition for 10 minutes in 1 nM of EDTA, pH 8.0, in a microwave oven. Sections were then washed twice in PBS and saturated with BSA 2% in PBS before staining with primary antibodies against: NB84a human neuroblastoma antigen (NB84a, Dako Italia SpA, Milan, Italy), Ki-67 proliferation antigen (Ki-67 mouse anti-human, Ki-55 clone, Dako), Smooth Muscle Actin (SMA) human antigen produced in mouse (1A4 clone, Dako) and CD34 transmembrane antigen produced in rat (CD34 anti-mouse, MEC14.7 clone, Immuno Tools GmbH, Friesoythe, Germany), as previously described (Pagnan et al., 2009; Pastorino et al., 2008). Anti-mouse IgG conjugated with Alexa Fluor 488 and anti-rabbit IgG conjugated with Alexa Fluor 555 were used as secondary antibodies (Invitrogen). TUNEL staining (i.e. labeling of DNA terminal fragments mediated by terminal deoxynucleotidyl transferase) was performed using a commercially available apoptosis detection kit (In situ Cell Death Detection, POD; Roche Molecular Biochemicals, Mannheim, Germany) according to the manufacturer's instructions, as previously described (Pagnan et al., 2009; Pastorino et al., 2003a). Samples were analyzed with Nikon E-1000 fluorescence microscope provided with filters specific for FITC and DAPI.

In addition, we performed immunohistochemistry against CD246-ALK human protein (ALK1 clone, Dako), matrix metalloproteinases 2 (MMP-2, 36006 clone, R & D System, Abingdon, United Kingdom) and 9 (MMP-9, 443 clone, R & D Systems) to detect the inhibition of tumor invasion and we used the antibody against Vascular Endothelium Growth Factor (VEGF) (Thermo Fisher Scientific, Fremont, Calif., USA) and VEGF receptor 2 (VEGF-R2) (89115 clone, R & D Systems) to detect the inhibition of tumor angiogenesis. Tissue sections (5 μm) were examined after staining with Mayer's hematoxylin-eosin (H&E) (Sigma).

Morphometric analysis was performed on 9 fields randomly selected every 3 sections, observed under Olympus photomicroscope at ×200 magnification, using Image Analysis software (Olympus, Italy). VEGF, VEGFR-2 and MMP-2/MMP-9 labeled areas were evaluated.

Analysis of Immune Responses

Peripheral blood polymorphonuclear cells (PBMC) were isolated from whole blood of healthy donors by density centrifugation with Ficoll-Histopaque Plus (Sigma). For immunostimulation tests, 3×10⁶ of freshly isolated PBMC were seeded in 12-well plates and submitted to culture in RPMI 1640 supplemented with 10% FBS. TL[siRNA] or siRNA not encapsulated in CCLs was added to cells at a final concentration of 100 nmol/L and culture supernatants were collected after 17 hours and analyzed in triplicate for Interferon-α (IFN-α) and Tumor Necrosis Factor-α (TNF-α) with ELISA kits for human Interferon-α and TNF-α (Bender Me-dSystems, GmbH, Wien, Austria). As positive control, PBMC were treated with 6 μg/ml of CpG oligonucleotides (CpG 2395, Coley Pharmaceutical, Ottawa, ON, Canada) and 100 ng/mL of lipopolysaccharides (LPS) for induction of IFN-α and TNF-α, respectively. Athymic mice were treated with 1.5 mg/kg of siRNA, either free or encapsulated in TL, for 24 hours, blood was collected through the retro-orbital sinus and then mice were sacrificed, as previously described. As positive control, mice were treated with 2.5 mg/kg of CpG. Serum was collected after coagulation and centrifugation, and ELISA tests were used to quantify IFN-α (PBL Biomedical Laboratories, New Brunswick, N.J., USA), IL1-α and TNF-α (Flowcytomix mouse, Bender MedSystems) according to the manufacturer's instructions.

Systemic Toxicity

In order to verify renal and hepatic toxicity, athymic mice were treated on alternate days for total 5 doses with 1.5 mg/kg of siRNA, either free or entrapped in TL, and blood samples were evaluated 3 hours after the first injection (acute toxicity) and one week after the treatment end (chronic toxicity) (FIG. 8) using commercial kits (Fisher Scientific Pittsburgh Pa.) according to the manufacturer's instructions.

Static methods

Results were expressed as confidence intervals around mean±95% (95% CI).

Statistical significance of differential results between experimental groups and controls was determined by ANOVA, with Tukey's multiple comparison test, using Graph-Pad Prism 3.0 software (GraphPad Software Inc., El Camino Real, San Diego, Calif.). Significance of differences among experimental groups (n=7-8/mice per group) in survival experiments was determined by Kaplan-Meier curves and Log-rank test (Graph-Pad Prism 3.0).

Results were considered significant if P value was <0.05.

All the publications mentioned in the description are included here as reference.

Even though the invention has been described with reference to specific embodiments, the invention is not be considered limited to these embodiments, but modifications to the modes for carrying out the invention clear for people skilled in the fields of biochemistry and molecular biology and similar technical fields have to be considered falling within the following claims.

### REFERENCES

Allen, T. M., and Cullis, P. R. (2004). Drug delivery systems: entering the mainstream. Science 303, 1818-1822.
Ambrogio, C., Martinengo, C., Voena, C., Tondat, F., Riera, L., di Celle, P. F., Inghirami, G., and Chiarle, R. (2009). NPM-ALK oncogenic tyrosine kinase controls T-cell identity by transcriptional regulation and epigenetic silencing in lymphoma cells. Cancer Res 69, 8611-8619.
C. Brignole, G. Pagnan, D. Marimpietri, E. Cosimo, T. M. Allen, M. Ponzoni and F. Pastorino. Targeted delivery system for antisense oligonucleotides: a novel experimental strategy for neuroblastoma treatment. Cancer Letters, 2003; 197: 231-235.
Brignole, C., Marimpietri, D., Pastorino, F., Di Paolo, D., Pagnan, G., Loi, M., Piccardi, F., Cilli, M., Tradori-Cappai, A., Arrigoni, G., et al. (2009). Anti-IL-10R antibody improves the therapeutic efficacy of targeted liposomal oligonucleotides. J Control Release 138, 122-127.
Brignole, C., Marimpietri, D., Pastorino, F., Nico, B., Di Paolo, D., Cioni, M., Piccardi, F., Cilli, M., Pezzolo, A., Corrias, M. V., et al. (2006). Effect of bortezomib on human neuroblastoma cell growth, apoptosis, and angiogenesis. J Natl Cancer Inst 98, 1142-1157.
Brignole, C., Pastorino, F., Marimpietri, D., Pagnan, G., Pistorio, A., Allen, T. M., Pistoia, V., and Ponzoni, M. (2004). Immune cell-mediated antitumor activities of GD2-targeted liposomal c-myb antisense oligonucleotides containing CpG motifs. J Natl Cancer Inst 96, 1171-1180.
Caren, H., Abel, F., Kogner, P., and Martinsson, T. (2008). High incidence of DNA mutations and gene amplifications of the ALK gene in advanced sporadic neuroblastoma tumours. Biochem J 416, 153-159.
Chambers, A. F., Groom, A. C., and MacDonald, I. C. (2002). Dissemination and growth of cancer cells in metastatic sites. Nat Rev Cancer 2, 563-572.
Chen, Y., Takita, J., Choi, Y. L., Kato, M., Ohira, M., Sanada, M., Wang, L., Soda, M., Kikuchi, A., Igarashi, T., et al. (2008). Oncogenic mutations of ALK kinase in neuroblastoma. Nature 455, 971-974.
Cheresh, D. A., Harper, J. R., Schulz, G., and Reisfeld, R. A. (1984). Localization of the gangliosides GD2 and GD3 in adhesion plaques and on the surface of human melanoma cells. Proc Natl Acad Sci USA 81, 5767-5771. 25
Chiarle, R., Voena, C., Ambrogio, C., Piva, R., and Inghirami, G. (2008). The anaplastic lymphoma kinase in the pathogenesis of cancer. Nat Rev Cancer 8, 11-23.
Cohn, S. L., Pearson, A. D., London, W. B., Monclair, T., Ambros, P. F., Brodeur, G. M., Faldum, A., Hero, B., Iehara, T., Machin, D., et al. (2009). The International Neuroblastoma Risk Group (INRG) classification system: an INRG Task Force report. J Clin Oncol 27, 289-297.
Corrias, M. V., Guarnaccia, F., and Ponzoni, M. (1997). Bioavailability of antisense oligonucleotides in neuroblastoma cells: comparison of efficacy among different types of molecules. J Neurooncol 31, 171-180.
Cosse, J. P., Ronvaux, M., Ninane, N., Raes, M. J., and Michiels, C. (2009). Hypoxia-induced decrease in p53 protein level and increase in c-jun DNA binding activity results in cancer cell resistance to etoposide. Neoplasia 11, 976-986.
Du, R., Lu, K. V., Petritsch, C., Liu, P., Ganss, R., Passegue, E., Song, H., Vandenberg, S., Johnson, R. S., Werb, Z., and Bergers, G. (2008). HlFlalpha induces the recruitment of bone marrow-derived vascular modulatory cells to regulate tumor angiogenesis and invasion. Cancer Cell 13, 206-220.
Fenske, D. B., and Cullis, P. R. (2008). Liposomal nanomedicines. Expert Opin Drug Deliv 5, 25-44.
George, R. E., Sanda, T., Hanna, M., Frohling, S., Luther, W., 2nd, Zhang, J., Ahn, Y., Zhou, W., London, W. B., McGrady, P., et al. (2008). Activating mutations in ALK provide a therapeutic target in neuroblastoma. Nature 455, 975-978.
Gesundheit, B., Moser, A., Or, R., and Klement, G. (2007). Successful antiangiogenic therapy for neuroblastoma with thalidomide. J Clin Oncol 25, 5321-5324.
Giles, R. V., Spiller, D. G., Grzybowski, J., Clark, R. E., Nicklin, P., and Tidd, D. M. (1998). Selecting optimal oligonucleotide composition for maximal antisense effect following streptolysin O-mediated delivery into human leukaemia cells. Nucleic Acids Res 26, 1567-1575.26
Gray, M. J., Van Buren, G., Dallas, N. A., Xia, L., Wang, X., Yang, A. D., Somcio, R. J., Lin, Y. G., Lim, S., Fan, F., et al. (2008). Therapeutic targeting of neuropilin-2 on colorectal carcinoma cells implanted in the murine liver. J Natl Cancer Inst 100, 109-120.
Handgretinger, R., Baader, P., Dopfer, R., Klingebiel, T., Reuland, P., Treuner, J., Reisfeld, R. A., and Niethammer, D. (1992). A phase I study of neuroblastoma with the anti-ganglioside GD2 antibody 14.G2a. Cancer Immunol Immunother 35, 199-204.
Janoueix-Lerosey, I., Lequin, D., Brugieres, L., Ribeiro, A., de Pontual, L., Combaret, V., Raynal, V., Puisieux, A., Schleiermacher, G., Pierron, G., et al. (2008). Somatic and germline activating mutations of the ALK kinase receptor in neuroblastoma. Nature 455, 967-970.
Li, Z., Tan, F., Liewehr, D. J., Steinberg, S. M., and Thiele, C. J. (2010). In Vitro and In Vivo Inhibition of Neuroblastoma Tumor Cell Growth by AKT Inhibitor Perifosine. J Natl Cancer Inst.102(11), 758-70.
Longo, L., Panza, E., Schena, F., Seri, M., Devoto, M., Romeo, G., Bini, C., Pappalardo, G., Tonini, G. P., and Perri, P. (2007). Genetic predisposition to familial neuroblastoma: identification of two novel genomic regions at 2p and 12p. Hum Hered 63, 205-211.
Lopes de Menezes, D. E., Pilarski, L. M., and Allen, T. M. (1998). In vitro and in vivo targeting of immunoliposomal doxorubicin to human B-cell lymphoma. Cancer Res 58, 3320-3330.
MacDiamid, J. A., Amaro-Mugridge, N. B., Madrid-Weiss, J., Sedliarou, I., Wetzel, S., Kochar, K., Brahmbhatt, V. N., Phillips, L., Pattison, S. T., Petti, C., et al. (2009). Sequential treatment of drug-resistant tumors with targeted minicells containing siRNAs or a cytotoxic drug. Nat Biotechnol 27, 643-651.
Marimpietri, D., Nico, B., Vacca, A., Mangieri, D., Catarsi, P., Ponzoni, M., and Ribatti, D. (2005. Synergistic inhibition of human neuroblastoma-related angiogenesis by vinblastine and rapamycin. Oncogene 24(45), 6785-95.
Maris, J. M., Hogarty, M. D., Bagatell, R., and Cohn, S. L. (2007). Neuroblastoma. Lancet 369, 2106-2120.
Matthay, K. K., Quach, A., Huberty, J., Franc, B. L., Hawkins, R. A., Jackson, H., Groshen, S.,Shusterman, S., Yanik, G., Veatch, J., et al. (2009). Iodine-131-metaiodobenzylguanidine double infusion with autologous stem-cell rescue for neuroblastoma: a new approaches to neuroblastoma therapy phase I study. J Clin Oncol 27, 1020-1025.
Merritt, W. M., Bar-Eli, M., and Sood, A. K. (2010). The dicey role of Dicer: implications for RNAi therapy. Cancer Res 70, 2571-2574.
Mosse, Y. P., Laudenslager, M., Longo, L., Cole, K. A., Wood, A., Attiyeh, E. F., Laquaglia, M. J., Sennett, R., Lynch, J. E., Perri, P., et al. (2008). Identification of ALK as a major familial neuroblastoma predisposition gene. Nature 455, 930-935.
Mosse, Y. P., Wood, A., and Maris, J. M. (2009). Inhibition of ALK signaling for cancer therapy. Clin Cancer Res 15, 5609-5614.
Mujoo, K., Cheresh, D. A., Yang, H. M., and Reisfeld, R. A. (1987). Disialoganglioside GD2 on human neuroblastoma cells: target antigen for monoclonal antibody-mediated cytolysis and suppression of tumor growth. Cancer Res 47, 1098-1104.
Mujoo Kalpana, Thomas J. Kipps, Hsin Ming Yang, David A.Cheresh, Ursula Wargalla, Diana J. Sander and Ralph A. Reisfeld (1989). Functional properties and Effect on Growth Suppression of Human Neuroblastoma Tumors by Isotype Switch Variants of Monoclonal Antiganglioside GD2 Antibody 14.18. Cancer Research 49, 2857-2861.
Muller, P. Y., Janovjak, H., Miserez, A. R., and Dobbie, Z. (2002). Processing of gene expression data generated by quantitative real-time RT-PCR. Biotechniques 32, 1372-1374, 1376, 1378-1379.
Nguyen, D. N., Chen, S.C., Lu, J., Goldberg,
M., Kim, P., Sprague, A., Novobrantseva, T., Sherman, J., Shulga-Morskaya, S., de Fougerolles, A., et al. (2009). Drug delivery-mediated control of RNA immunostimulation. Mol Ther 17, 1555-1562.
Pagnan, G., Di Paolo, D., Carosio, R., Pastorino, F., Marimpietri, D., Brignole, C., Pezzolo, A., Loi, M., Galietta, L. J., Piccardi, F., et al. (2009). The combined therapeutic effects of bortezomib and fenretinide on neuroblastoma cells involve endoplasmic reticulum stress response. Clin Cancer Res 15, 1199-1209.
Pagnan, G., Montaldo, P. G., Pastorino, F., Raffaghello, L., Kirchmeier, M., Allen, T. M., and Ponzoni, M. (1999). GD2-mediated melanoma cell targeting and cytotoxicity of liposomeentrapped fenretinide. Int J Cancer 81, 268-274.
Pagnan, G., Stuart, D. D., Pastorino, F., Raffaghello, L., Montaldo, P. G., Allen, T. M., Calabretta, B., and Ponzoni, M. (2000). Delivery of c-myb antisense oligodeoxynucleotides to human neuroblastoma cells via disialoganglioside GD(2)-targeted immunoliposomes: antitumor effects. J Natl Cancer Inst 92, 253-261.
Palmer, R. H., Vernersson, E., Grabbe, C., and Hallberg, B. (2009). Anaplastic lymphoma kinase: signalling in development and disease. Biochem J 420, 345-361.
Passoni, L., Longo, L., Collini, P., Coluccia, A. M., Bozzi, F., Podda, M., Gregorio, A., Gambini, C., Garaventa, A., Pistoia, V., et al. (2009). Mutation-Independent Anaplastic Lymphoma Kinase Overexpression in Poor Prognosis Neuroblastoma Patients. Cancer Res 8(2): 129-38.
Pastorino, F., Brignole, C., Di Paolo, D., Nico, B., Pezzolo, A., Marimpietri, D., Pagnan, G., Piccardi, F., Cilli, M., Longhi, R., et al. (2006). Targeting liposomal chemotherapy via both tumor cell-specific and tumor vasculature-specific ligands potentiates therapeutic efficacy. Cancer Res 66, 10073-10082.
Pastorino, F., Brignole, C., Marimpietri, D., Cilli, M., Gambini, C., Ribatti, D., Longhi, R., Allen,T. M., Corti, A., and Ponzoni, M. (2003a). Vascular damage and anti-angiogenic effects of tumor vessel-targeted liposomal chemotherapy. Cancer Res 63, 7400-7409.
Pastorino, F., Brignole, C., Marimpietri, D., Pagnan, G., Morando, A., Ribatti, D., Semple, S.C., Gambini, C., Allen, T. M., and Ponzoni, M. (2003b). Targeted liposomal c-myc antisense oligodeoxynucleotides induce apoptosis and inhibit tumor growth and metastases in human melanoma models. Clin Cancer Res 9, 4595-4605.
Pastorino, F., Brignole, C., Marimpietri, D., Sapra, P., Moase, E. H., Allen, T. M., and Ponzoni, M. (2003c). Doxorubicin-loaded Fab′ fragments of anti-disialoganglioside immunoliposomes selectively inhibit the growth and dissemination of human neuroblastoma in nude mice. Cancer Res 63, 86-92.
Pastorino, F., Di Paolo, D., Loi, M., Becherini, P., Caffa, I., Zorzoli, A., Marimpietri, D., Carosio, R., Perri, P., Montaldo, P. G., et al. (2009). Recent advances in targeted anti-vasculature therapy: the neuroblastoma model. Curr Drug Targets 10, 1021-1027.
Pastorino, F., Di Paolo, D., Piccardi, F., Nico, B., Ribatti, D., Daga, A., Baio, G., Neumaier, C. E., Brignole, C., Loi, M., et al. (2008). Enhanced antitumor efficacy of clinical-grade vasculaturetargeted liposomal doxorubicin. Clin Cancer Res 14, 7320-7329.
Pastorino, F., Marimpietri, D., Brignole, C., Di Paolo, D., Pagnan, G., Daga, A., Piccardi, F., Cilli, M., Allen, T. M., and Ponzoni, M. (2007). Ligand-targeted liposomal therapies of neuroblastoma. Curr Med Chem 14, 3070-3078.
Pastorino, F., Stuart, D., Ponzoni, M., and Allen, T. M. (2001). Targeted delivery of antisense oligonucleotides in cancer. J Control Release 74, 69-75.
Pastorino F, Loi M, Sapra P, Becherini P, Cilli M, Emionite L, Ribatti D, Greenberger L M, Horak I D, Ponzoni M. Tumor Regression and Curability of Preclinical Neuroblastoma Models by PEGylated SN38 (EZN-2208), a Novel Topoisomerase I Inhibitor. Clin Cancer Res. 2010 Oct. 1; 16(19):4809-4821. Epub 2010 Aug. 11.
Piva, R., Chiarle, R., Manazza, A. D., Taulli, R., Simmons, W., Ambrogio, C., D'Escamard, V., Pellegrino, E., Ponzetto, C., Palestro, G., and Inghirami, G. (2006a). Ablation of oncogenic ALK is a viable therapeutic approach for anaplastic large-cell lymphomas. Blood 107, 689-697.
Piva, R., Pellegrino, E., Mattioli, M., Agnelli, L., Lombardi, L., Boccalatte, F., Costa, G., Ruggeri, B. A., Cheng, M., Chiarle, R., et al. (2006b). Functional validation of the anaplastic lymphoma kinase signature identifies CEBPB and BCL2A1 as critical target genes. J Clin Invest 116, 3171-3182.
Raffaghello, L., Marimpietri, D., Pagnan, G., Pastorino, F., Cosimo, E., Brignole, C., Ponzoni, M., and Montaldo, P. G. (2003a). Anti-GD2 monoclonal antibody immunotherapy: a promising strategy in the prevention of neuroblastoma relapse. Cancer Lett 197, 205-209.
Raffaghello, L., Pagnan, G., Pastorino, F., Cosimo, E., Brignole, C., Marimpietri, D., Montaldo, P. G., Gambini, C., Allen, T. M., Bogenmann, E., and Ponzoni, M. (2003b). In vitro and in vivo antitumor activity of liposomal Fenretinide targeted to human neuroblastoma. Int J Cancer 104, 559-567.
Reimer, D. L., Zhang, Y., Kong, S., Wheeler, J. J., Graham, R. W., and Bally, M. B. (1995). Formation of novel hydrophobic complexes between cationic lipids and plasmid DNA. Biochemistry 34, 12877-12883.
Ribatti, D., and Ponzoni, M. (2005). Antiangiogenic strategies in neuroblastoma. Cancer Treat Rev 31, 27-34.
Ribatti, D., Vacca, A., Nico, B., De Falco, G., Giuseppe Montaldo, P., and Ponzoni, M. (2002). Angiogenesis and anti-angiogenesis in neuroblastoma. Eur J Cancer 38, 750-757.
Rossler, J., Taylor, M., Geoerger, B., Farace, F., Lagodny, J., Peschka-Suss, R., Niemeyer, C. M., and Vassal, G. (2008). Angiogenesis as a target in neuroblastoma. Eur J Cancer 44, 1645-1656.
Rozen, S., and Skaletsky, H. (2000). Primer3 on the WWW for general users and for biologist programmers. Methods Mol Biol 132, 365-386.
Santos, A. O., da Silva, L. C., Bimbo, L. M., de Lima, M. C., Simoes, S., and Moreira, J. N. (2010). Design of peptide-targeted liposomes containing nucleic acids. Biochim Biophys Acta 1798, 433-441.
Schulz, G., Cheresh, D. A., Varki, N. M., Yu, A., Staffileno, L. K., and Reisfeld, R. A. (1984). Detection of ganglioside GD2 in tumor tissues and sera of neuroblastoma patients. Cancer Res 44, 5914-5920.
Stuart, D. D., Kao, G. Y., and Allen, T. M. (2000). A novel, long-circulating, and functional liposomal formulation of antisense oligodeoxynucleotides targeted against MDR1. Cancer Gene Ther 7, 466-475.
Tanaka, T., Mangala, L. S., Vivas-Mejia, P. E., Nieves-Alicea, R., Mann, A. P., Mora, E., Han, H. D., Shahzad, M. M., Liu, X., Bhavane, R., et al. (2010). Sustained small interfering RNA delivery by mesoporous silicon particles. Cancer Res 70, 3687-3696.
Taulli, R., Accornero, P., Follenzi, A., Mangano, T., Morotti, A., Scuoppo, C., Formi, P. E., Bersani, F., Crepaldi, T., Chiarle, R., et al. (2005). RNAi technology and lentiviral delivery as a powerful tool to suppress Tpr-Met-mediated tumorigenesis. Cancer Gene Ther 12, 456-463.
Verma, D., Kantarjian, H., Shan, J., O'Brien, S., Estrov, Z., Garcia-Manero, G., Koller, C., Borthakur, G., and Cortes, J. (2010). Survival outcomes for clonal evolution in chronic myeloid leukemia patients on second generation tyrosine kinase inhibitor therapy. Cancer 116, 2673-2681.
Webb, T. R., Slavish, J., George, R. E., Look, A. T., Xue, L., Jiang, Q., Cui, X., Rentrop, W. B., and Morris, S. W. (2009). Anaplastic lymphoma kinase: role in cancer pathogenesis and smallmolecule inhibitor development for therapy. Expert Rev Anticancer Ther 9, 331-356.
Whitehead, K. A., Langer, R., and Anderson, D. G. (2009). Knocking down barriers: advances in siRNA delivery. Nat Rev Drug Discov 8, 129-138. Yu, A. L., Uttenreuther-Fischer, M. M., Huang, C. S., Tsui, C. C., Gillies, S. D., Reisfeld, R. A., and Rung, F. H. (1998). Phase I trial of a human-mouse chimeric anti-disialoganglioside monoclonal antibody ch14.18 in patients with refractory neuroblastoma and osteosarcoma. J Clin Oncol 16, 2169-2180.
Yuan, F., Leunig, M., Huang, S. K., Berk, D. A., Papahadjopoulos, D., and Jain, R. K. (1994). Microvascular permeability and interstitial penetration of sterically stabilized (stealth) liposomes in a human tumor xenograft. Cancer Res 54, 3352-3356.
Zimmermann, T. S., Lee, A. C., Akinc, A., Bramlage, B., Bumcrot, D., Fedoruk, M. N., Harborth, J., Heyes, J. A., Jeffs, L. B., John, M., et al. (2006). RNAi-mediated gene silencing in non-human primates. Nature 441, 111-114.

The invention claimed is:

1. A lipid vector for the transfer into target cells of at least one therapeutic agent comprising:
at least one recognizing molecule directed against a cell target molecule carried on an external surface of said lipid vector,
wherein said recognizing molecule is directed against an epitope of GD₂ disialoganglioside, a tumor-associated membrane antigen expressed abundantly by neuroectodermal tumor cells, and
wherein said lipid vector contains, as therapeutic agent, double-stranded RNA synthetic oligonucleotides, complementary to mRNA (messenger RNA) and coding for at least one gene with oncogenic function, such to obtain silencing, that is the and selectively decreasing expression of a specific gene in target cells.

2. The lipid vector according to claim 1, wherein said synthetic oligonucleotides are specific for an ALK gene.

3. The lipid vector according to claim 1, wherein said synthetic oligonucleotides comprise a siRNA (small interfering RNA) having a sequence that presents one or both of chemical or physical modifications of nucleotides, and wherein said modifications increase stability of hybrids with mRNA or DNA or increase stability to enzymatic degradation by cellular nucleases, implement specificity and efficacy, or avoid triggering an immune response.

4. The lipid vector according to claim 3, wherein said modification affects LNA (Locked Nucleic Acid) nucleotides and comprise a methylenic bridge between 2′-O (oxygen) and 4′-C (carbon) atoms.

5. The lipid vector according to claim 3, wherein said modification affects 2′-O-Methyl-ribonucleotides, which present a substitution of hydrogen of a hydroxyl group (OH) at position 2′ with a methyl group (CH₃).

6. The vector according to claim 1,
wherein the lipid vector is a liposome having one or more cationic lipids, neutral lipids, or PEG-lipids, comprising lipids with polyethyleneglycol (PEG) chains that include hydrogenated soybean phosphatidylcholine (HSPC), cholesterol (CHE),1,2-distearoyl-glycero-3-phosphoethanolamine-N-[methoxy(polyethylene glycol)-₂₀₀₀] (DSPE-PEG₂₀₀₀)/1,2-distearoyl-sn-glycero-3-phosphoethanolamine-N-[methoxy(polyethylene glycol)-₂₀₀₀] modified with a maleimidic group in the distal end of the chain (1,2-distearoyl-sn-glycero-3-phosphoethanolamine-N-[maleimide (polyethylene glycol)-₂₀₀₀], DSPE-PEG₂₀₀₀-MAL, or 1,2-dioleoyl-1-3-trimethylammonium propane (DOTAP),
wherein said synthetic oligonucleotides are complexed with cationic lipids that are coated with neutral lipids thus forming a coated cationic liposome, and
wherein negative to positive charge ratio between said synthetic oligonucleotides and said lipids is 1:1 and cholesterol to phospholipids molecular ratio is 1:2.

7. The lipid vector according to claim 1, wherein the at least one recognizing molecule is an anti-GD₂ monoclonal antibody or a fragment thereof (Fab′), and wherein said at least one recognizing molecule has been coupled with a maleimidic end of DSPE-PEG₂₀₀₀-MAL, a lipid which is part of a neutral external layer of a vesicle.

8. The vector according to claim 7, wherein a diameter of the lipid vector is 100-200 nm, with a mean of 135-140 nm, is increased by about 10-30 nm by presence of a recognizing molecule of GD₂ disialoganglioside, with 0.1±0.025 polydispersity and mean coupling efficiency of 50±15 μg whole antibody/μmol phospholipids, or of 30±15 g antibody reduced to fragments of a anti GD₂ antibody/μmol phospholipids.

9. The lipid vector according claim 1, wherein the lipid vector has a loading capacity is about 10 nmol siRNA/μmol of total lipids.

10. The lipid vector according to claim 1, wherein the at least one recognizing molecule recognizes a disease-associated target molecule different from GD₂ disialoganglioside.

11. The lipid vector according to claim 1, wherein the lipid vector contains a therapeutic agent different from sequence-specific siRNA for ALK gene.

12. The lipid vector according to claim 1, wherein the target cells are neuroectodermal tumor cells.

13. The lipid vector according to claim 1, wherein the target cells are cells of a large cell anaplastic lymphoma, lung tumor, rhabdomyosarcomas or of an ectodermal, myofibroblastic or neuroblastic solid tumor.

14. A composition comprising:
at least one lipid vector according to claim 1.

15. The composition according to claim 14, wherein said composition comprises at least two lipid vectors, and wherein at least one of the two lipid vectors comprises a therapeutic agent different from ALK-specific siRNAs, or carries a recognizing molecule having a molecule different from GD₂ disialoganglioside as target.

16. The composition according to claim 14, wherein the at least one lipid vector is provided in combination with a second further therapeutic agent, natural or synthetic, free or contained within viral or non-viral vectors.

17. The composition claim 16, wherein the second therapeutic agent is adapted for treating cancer or phenomena associated therewith.

18. A method of treatment comprising:
providing a lipid vector according to claim 1; and
administering said lipid vector to a patient having a tumor.

19. The method according to claim 18, wherein the tumor is a neuroectodermal tumor, a neuroblastoma, a melanoma, a neurosarcoma or a phenomenon associated therewith.

20. The method according to claim 18, wherein the lipid vector contains a siRNA (small interfering RNA) for silencing an ALK oncogene at any gene status, such to inhibit cell proliferation, induce apoptosis, or decrease density of blood vessels.

21. (canceled)

22. The method according to claim 20, wherein the siRNA is adapted to inhibit a MAP-kinase or MAPK pathway and a consequent decrease in ERK1/2 kinase phosphorylation, to inhibit inhibition a HIF-1α signal pathway and a consequent decrease in production of Vascular Endothelium Growth Factor (VEGF) and its receptor VEGFR-2, or to inhibit endothelial and perivascular cells with a decreased expression of CD34 and SMA proteins, correlated with angiogenesis, and of MMP-2 and MMP-9 metalloproteinases, correlated with tumor invasiveness.

23. The method according to claim 18, wherein the lipid vector comprises a vesicle that contains a cationic fluid and is coated with a neutral lipid, and wherein the lipid vector is provided with a recognizing molecule targeting a membrane molecule on target cells and transferring sequence-specific oligonucleotides, such to protect siRNAs, microRNAs (miRNAs), miRNA mimics, or miRNA antagonists against degradation by nucleases, to increment stability and thus activity of siRNAs, microRNAs (miRNAs), miRNA mimics, or miRNA antagonists, to increment membrane permeability and specific cellular uptake, or to avoid innate immune response mediated by siRNAs, microRNAs (miRNAs), miRNA mimics, miRNA antagonists from being activated.

24. A method of inducing the arrest of cell proliferation, inducing apoptosis, or inhibiting angiogenesis, comprising:
transferring molecules mediating RNAi (RNA interference) into specific target cells through a non-viral lipid vector according to claim 1 or a composition comprising said lipid vector, thereby obtaining a silencing of said ALK gene.

25. The method according to claim 24, further comprising one or more of the following steps:
inhibiting MAP-kinase or MAPK pathway and consequently decreasing in ERK1/2 kinase phosphorylation;
inhibiting a HIF-1α signal pathway and consequently decreasing production of Vascular Endothelium Growth Factor (VEGF) and its receptor VEGFR-2; or
inhibiting endothelial and perivascular cells highlighted respectively by CD34 and SMA proteins, correlated with angiogenesis, and of MMP-2 and MMP-9 metalloproteinases, correlated with tumor invasiveness.

26. The method according to claim 24, further comprising the step of administering said lipid vector or a composition containing said lipid vector, alone or in combination with another therapeutic agent or compound.

27. The method according to claim 26, wherein the step of administering comprises administering intratumorally, intravenously or subcutaneously.

28. (canceled)

29. The lipid vector according to claim 1, wherein said synthetic oligonucleotides comprise siRNAs (small interfering RNAs).

30. The lipid vector according to claim 3, wherein said nucleotides comprise one or both of LNAs (Locked Nucleic Acid) or 2′-O-Methyl-ribonucleotides.

31. The lipid vecor according to claim 11, wherein said sequence-specific siRNA for ALK gene, comprises a sequence-specific synthetic oligonucleotide, a siRNA specific for a different gene, a single-stranded antisense oligonucleotide provided with a CpG island, a microRNA (miRNA), a miRNA mimic, a miRNA antagonist, an anticancer drug, an anti-inflammatory drug, or an anti-angiogenic drug.

32. The composition according to claim 16, wherein said second therapeutic agent is an antibody, a protein inhibitor, or a chemotherapeutic agent.

33. The method according to claim 24, wherein transferring molecules mediating RNAi comprises transferring an ALK gene-specific siRNA.
